(19) United States
(12) Patent Application Publication
(10) Pub. No.: US 2024/0156846 A1
(43) Pub. Date: May 16, 2024

(54) COMPOSITIONS AND METHODS FOR TREATING NEURODEGENERATIVE DISORDERS

(71) Applicants: Arizona Board of Regents on Behalf of the University of Arizona, Tucson, AZ (US); THE TRUSTEES OF COLUMBIA UNIVERSITY IN THE CITY OF NEW YORK, New York, NY (US)

(72) Inventors: Rui Chang, Tucson, AZ (US); Patrick T. Ronaldson, Tucson, AZ (US); Qianying He, Tucson, AZ (US); Wassim ELYAMAN, New York, NY (US); Elizabeth BRADSHAW, New York, NY (US); Kuixi ZHU, Tucson, AZ (US)

(21) Appl. No.: 18/113,981

(22) Filed: Feb. 24, 2023

Related U.S. Application Data
(63) Continuation-in-part of application No. PCT/US2021/048034, filed on Aug. 27, 2021.
(60) Provisional application No. 63/071,035, filed on Aug. 27, 2020.
(60) Provisional application No. 63/071,032, filed on Aug. 27, 2020.

(51) Int. Cl. A61K 31/7028; A61K 31/26; A61K 31/357; A61K 31/404; A61K 31/4409; A61K 31/5375; A61K 45/06; A61P 25/28

(52) U.S. Cl. CPC A61K 31/7028; A61K 31/26; A61K 31/357; A61K 31/404; A61K 31/4409; A61K 31/5375; A61K 45/06; A61P 25/28

(57) ABSTRACT

This invention relates generally to neurodegenerative diseases and conditions (e.g., Alzheimer's disease) characterized with dysfunctional energetic function, unregulated microglia phagocytic activity and other related de-regulated biological functions. This invention further relates to methods and compositions for treating such neurodegenerative diseases and conditions with pharmaceutical compositions capable of protecting neurons from cell death and unregulated microglia phagocytic activity.

### CROSS-REFERENCE TO RELATED APPLICATIONS

5 This application is a continuation-in-part of International Patent Application No. PCT/US2021/048034, filed Aug. 27, 2021, which claims the benefit of U.S. Prov. Appl. 63/071,035, filed Aug. 27, 2020, and U.S. Provisional Patent Application No. 63/071,032, filed Aug. 27, 2020, the contents of which are incorporated herein by reference in their entireties.

### STATEMENT REGARDING FEDERALLY SPONSORED RESEARCH OR DEVELOPMENT

This invention was made with government support under Grant Nos AG062620 and AG057457 awarded by National Institutes of Health. The government has certain rights in the invention.

### FIELD OF THE INVENTION

This invention relates generally to neurodegenerative diseases and conditions (e.g., Alzheimer's disease) characterized with dysfunctional energetic function, unregulated microglia phagocytic activity and other related de-regulated biological functions. This invention further relates to methods and compositions for treating such neurodegenerative diseases and conditions with pharmaceutical compositions capable of protecting neurons from cell death and unregulated microglia phagocytic activity.

### BACKGROUND OF THE INVENTION

There is an urgent need to develop novel therapies for neurodegenerative diseases and conditions such as Alzheimer's disease (AD). 10% of persons over age 65 and up to 50% over age 85 have dementia, with over 30 million people affected worldwide. AD affects over 26 million people worldwide and currently there is no cure for the disease. With the growing number of people living to older ages, there is an urgency to better understand elements of the pathogenic pathway, discover agents that target these elements, and establish their roles in the treatment and prevention of AD.

As such, improved methods for treating neurodegenerative disorders (e.g., AD) are needed.

The present invention addresses this need.

### SUMMARY

Alzheimer's disease (AD) is the most common neurodegenerative disease with no effective treatment to date. The onset and development of AD pathology involves complex biological processes, leading to the formation of neurite plaques and neurofibrillary tangles (NFTs) and which are the pathological hallmarks of AD. Past AD drug developments have been overwhelmingly unsuccessful as the prevalence of the disease rises throughout the world, it becomes ever more crucial to develop disease-modifying drugs for AD.

In experiments conducted during the course of developing embodiments for the present invention, the inventors showed that by developing an integrative and translational drug repurposing pipeline to analyze multi-omics data generated from post-mortem brain tissues in Accelerating Medicines Partnership (AMP)-AD, three natural compounds (NAD⁺, NADH, PEITC) appear to successfully perturb the pathological hallmarks of the disease by modulating multiple upstream cellular pathways linked to AD rather than targeting a singular pathway. The innovative pipeline centered on the construction of de-novo causal genetic regulatory network to discover key driver and pathways associated with AD by using a cutting-edge predictive (causal) network model. The in vitro experiments with human primary neurons and microglial cells demonstrate significant neuroprotective and phagocytosis-promoting effects of the three compounds and their combinations (NADH/PEITC and NAD⁺/PEITC) demonstrated strong synergistic therapeutic effects under AD condition. The brain proteomics data in 3xTg AD mice treated with individual compound further revealed different pathways, involving mitochondrial energy metabolism, immune responses, and phagocytosis, were shown to be significantly perturbed. Moreover, 3xTg mice treated with NADH, NAD+ and PEITC respectively showed significantly reduced the neurotoxic ratios of Aβ2 to 40 and p231-tau to total tau, and humanized-Tau (hTau) mice treated with PEITC showed significant reduction of neurotoxic ratio of p231-tau to total tau in the brain. These natural compounds individually or in combination are indicated as preventative and therapeutic agents for combating AD.

Accordingly, the present invention relates generally to neurodegenerative diseases and conditions (e.g., Alzheimer's disease) characterized with dysfunctional energetic function, unregulated microglia phagocytic activity and other related de-regulated biological functions. This invention further relates to methods and compositions for treating such neurodegenerative diseases and conditions with pharmaceutical compositions comprising one or more

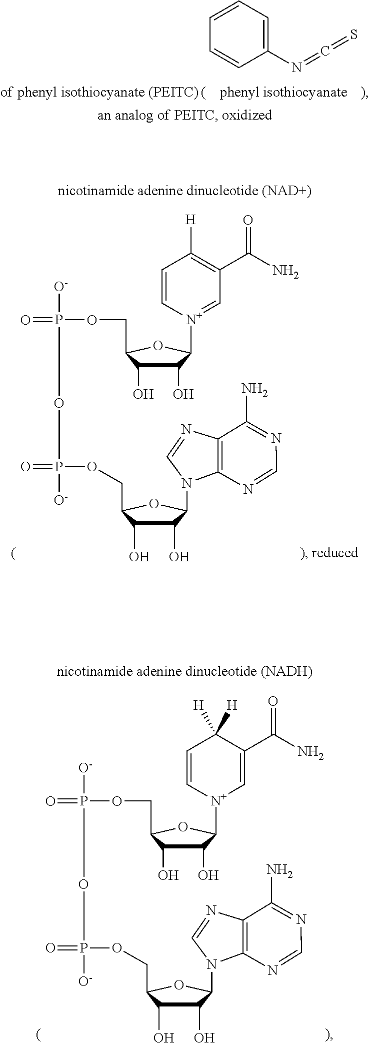

nicotinamide mononucleotide (NMN), nicotinamide riboside (NR), a chemical moiety comprising isothiocyanate (e.g., 7-methylsulfinylheptyl, 8-methylsulfinyloctyl), vitamin c (ascorbate acid), glutathione, and myrosinase, wherein such compositions are capable of protecting neurons from cell death and unregulated microglia phagocytic activity.

In certain embodiments, the present invention provides a method of treating a mammal suffering from a condition characterized with dysfunctional energetic function, unregulated microglia phagocytic activity, and other related de-regulated biological functions comprising administering to the mammal a composition comprising one or more agents capable of protecting neurons from cell death and unregulated microglia phagocytic activity.

In certain embodiments, the present invention provides a method for preventing and/or inhibiting neuronal cell death in a mammal in need thereof, the method comprising administering to the mammal a composition comprising one or more agents capable of protecting neurons from cell death and unregulated microglia phagocytic activity.

In certain embodiments, the present invention provides a method for preventing and/or inhibiting unregulated microglia phagocytic activity in a mammal in need thereof, the method comprising administering to the mammal a composition comprising one or more agents capable of protecting neurons from cell death and unregulated microglia phagocytic activity.

In some embodiments, the condition characterized with dysfunctional energetic function, unregulated microglia phagocytic activity, and other related de-regulated biological functions is one or more of a neurodegenerative disorder, aging, systemic inflammation, neuroinflammation, cancer, and diabetes.

In some embodiments, the neurodegenerative disorder selected from AD, Parkinson's disease, Huntington's disease, amyotrophic lateral sclerosis, motor neuron disease, subjective memory complaints, and mild cognitive impairment (MCI). In some embodiments, the AD is an early stage, prodromal phase of AD or late stage.

In some embodiments, the mammal is a human patient.

In certain embodiments, the present invention provides a method for preventing and/or inhibiting neuronal cell death in a subject suffering from a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) comprising, consisting of, or consisting essentially of administering to the subject a therapeutically effective amount of one or more agents capable of preventing and/or inhibiting unregulated microglia phagocytic activity.

In certain embodiments, the present invention provides for preventing and/or inhibiting unrelated microglia phagocytic activity in neuronal cells of a subject suffering from a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) comprising, consisting of, or consisting essentially of administering to the subject a therapeutically effective amount of one or more agents capable of preventing and/or inhibiting unregulated microglia phagocytic activity.

In certain embodiments, the present invention provides a method of preventing the onset of a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) in a subject (e.g., a human subject) comprising, consisting of, or consisting essentially of administering to the subject a therapeutically effective amount of one or more agents capable of preventing and/or inhibiting unregulated microglia phagocytic activity.

In certain embodiments, the present invention provides a method of treating and/or ameliorating the symptoms of a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) in a subject (e.g., a human subject) comprising, consisting of, or consisting essentially of administering to the subject a therapeutically effective amount of one or more agents capable of preventing and/or inhibiting unregulated microglia phagocytic activity.

The compositions, methods, systems, and kits described herein are not limited to a specific type or kind of agent capable of protecting neurons from cell death and unregulated microglia phagocytic activity. In some embodiments, the agent is selected from PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the agent is a pharmaceutical composition comprising a therapeutically effective amount of one or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase.

In some embodiments, the agent capable of protecting neurons from cell death and unregulated microglia phagocytic activity is derived from natural plants and seeds, and their extracts or derivatives. In some embodiments, the agent capable of protecting neurons from cell death and unregulated microglia phagocytic activity is derived from watercress, Cruciferous Vegetables, mustard, white mustard (Sinapis alba), garden cress (Lepidium sativum), wasabi (Wasabia japonica), and daikon (Raphanus sativus In some embodiments, the agent capable of protecting neurons from cell death and unregulated microglia phagocytic activity is derived from members of the family Brassicaceae, including yellow mustard (Brassica juncea), rape seed (Brassica napus). and common dietary Brassicas including, but not limited to, broccoli, cauliflower, cabbage, bok choy, kale, Papaya seeds, and cabbage aphid.

The compositions, methods, systems, and kits described herein are not limited to a specific type or kind of PEITC analog.

In some embodiments, a PEITC analog is any chemical moiety related to watercress and/or other cruciferous plant extraction and the structures related to PEITC.

In some embodiments, the PEITC analog is selected from

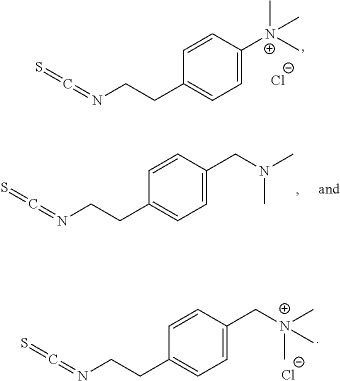 [SMILES: C[N+](C)(C)c1ccc(CCN=C=S)cc1 ; [Cl-] ; CN(C)Cc1ccc(CCN=C=S)cc1 ; C[N+](C)(C)Cc1ccc(CCN=C=S)cc1 ; [Cl-]]

In some embodiments, the PEITC analog is selected from:

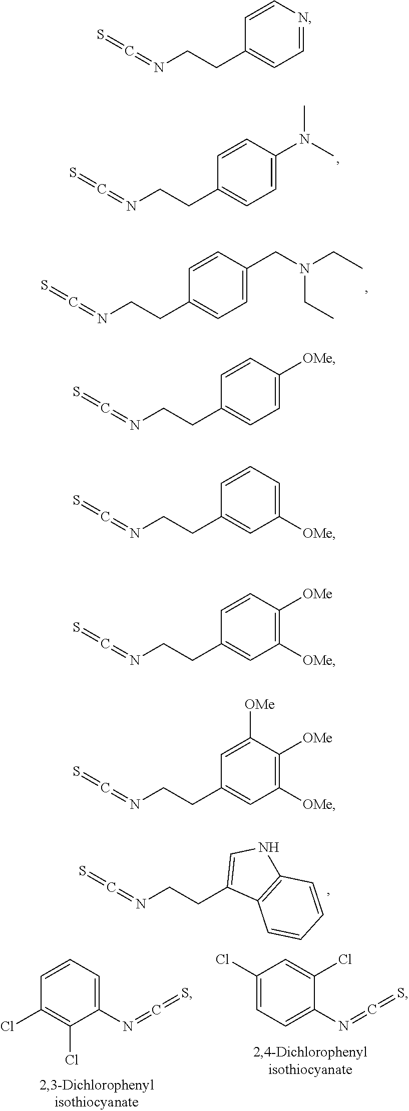

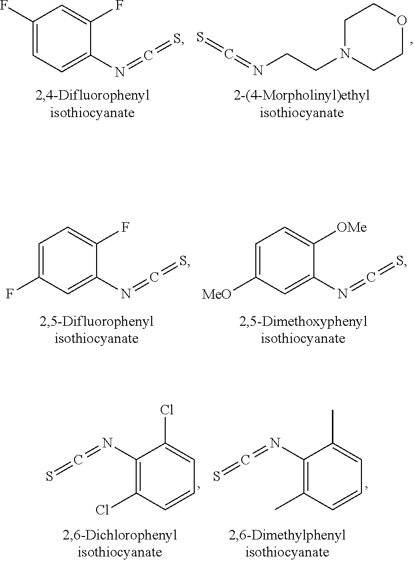

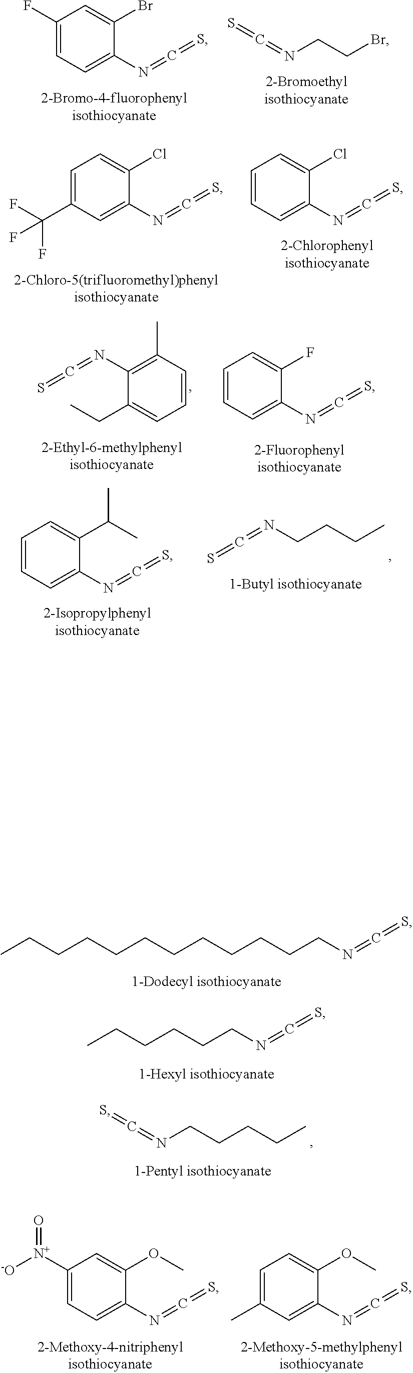

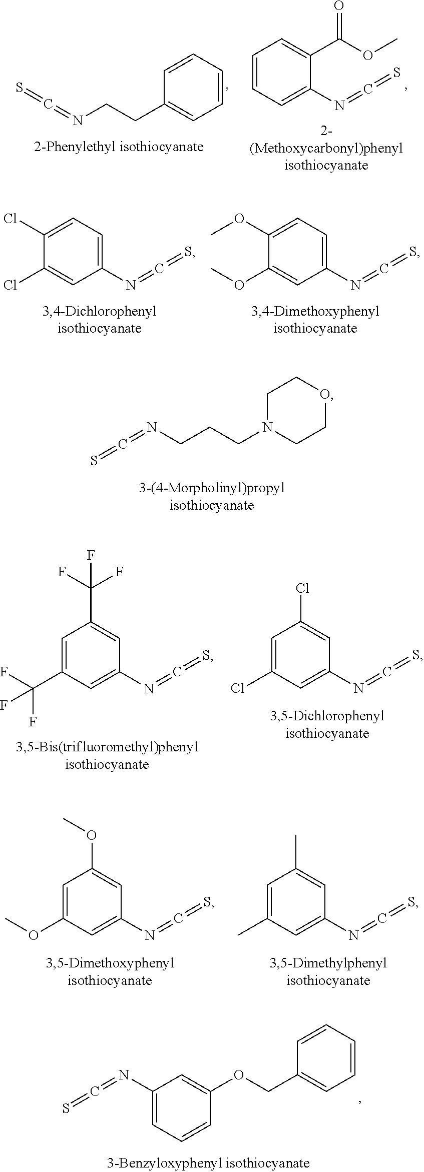

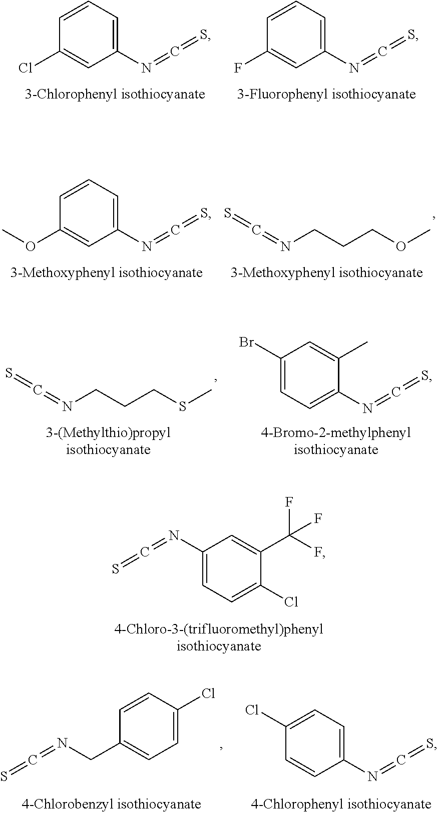

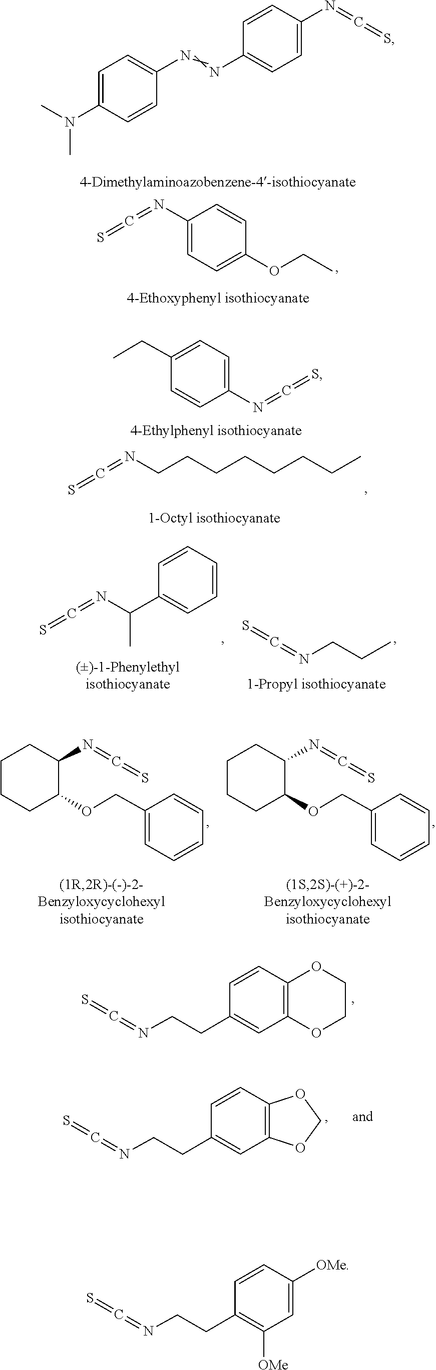

In some embodiments, the PEITC analog is selected from:

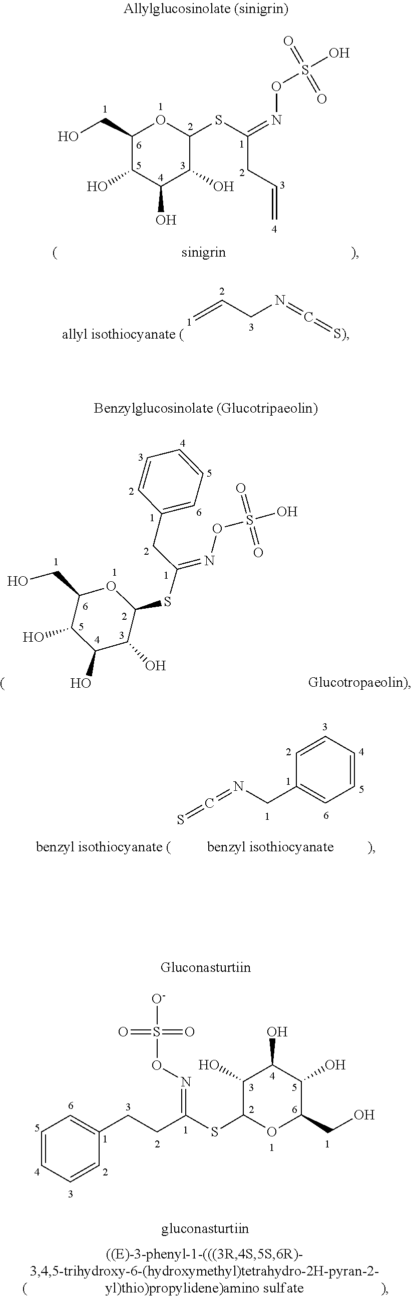

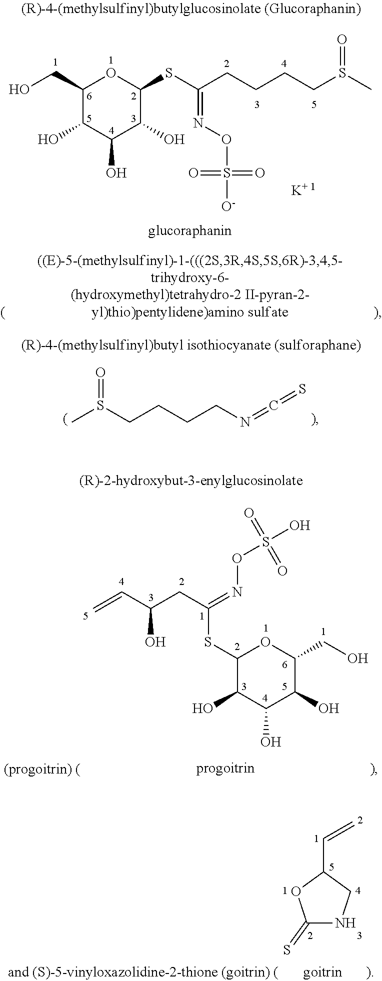

In certain embodiments, the one or more agents capable of protecting neurons from cell death and unregulated microglia phagocytic activity may be comprised within any type or kind of composition. For example, in some embodiments, such a composition may be an over-the-counter composition, a pharmaceutical composition, or any kind of cosmetic composition.

In certain embodiments, the present provides the following compounds:

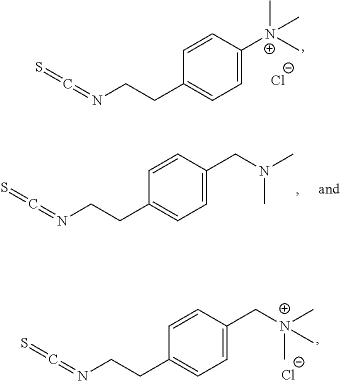 [SMILES: C[N+](C)(C)c1ccc(CCN=C=S)cc1 ; [Cl-] ; CN(C)Cc1ccc(CCN=C=S)cc1 ; C[N+](C)(C)Cc1ccc(CCN=C=S)cc1 ; [Cl-]]

including pharmaceutically acceptable salts, solvates, and/or prodrugs thereof.

In certain embodiments, the present invention provides a composition comprising one or more of the following: PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises two or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises three or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition is an over-the-counter composition, or a pharmacological prescription.

In certain embodiments, the present invention provides an over-the-counter composition comprising one or more of the following: PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises two or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises three or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the over-the-counter composition is a tablet, capsule, powder, suspension, or solution.

### BRIEF DESCRIPTION OF THE DRAWINGS

FIG. 1 depicts a translational drug-repurposing pipeline described herein (see, Example I).

FIG. 2 shows robust and significantly up- and down-regulated genes/proteins associated with AD (see, Example I).

FIG. 3 summarizes tested allelic loci-transcript and allelic loci-peptide correlation across brain regions/cell types and cohorts.

FIG. 4 depicts relationship between NADH, NAD+, PEITC and 138 key drivers (genes and peptides) for AD (see, Example I).

FIG. 5 depicts graphical data demonstrating the calculations utilized to obtain the data described in Example I.

FIGS. 6A-B depicts graphical data demonstrating the calculations utilized to obtain the data described in Example I.

FIGS. 7A-B depicts graphical data demonstrating the calculations utilized to obtain the data described in Example I.

FIGS. 8A-B depicts graphical data demonstrating the calculations utilized to obtain the data described in Example I.

FIGS. 9A-D depicts graphical data demonstrating the calculations utilized to obtain the data described in Example I.

### DEFINITIONS

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising/* or “having” certain elements are also contemplated as “consisting essentially of and “consisting of those certain elements.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “over-the-counter” means to provide by retail purchase without a prescription or license from a physician or medical practitioner (e.g., does not require a prescription from a physician in order to be administered to the human).

As used herein, the term “pharmaceutical compound” refers to any physical state of a material. Pharmaceutical compounds include but are not limited to capsules, tablets, liquids, topical formulations, and inhaled formulations.

Unless otherwise defined, all technical terms used herein have the same meaning as commonly understood by one of ordinary skill in the art to which this disclosure belongs.

### DETAILED DESCRIPTION

AD is a pressing global problem effecting over 40 million people currently living around the world (Collaborators GBDD, 2019). Furthermore, the number of individuals with AD continues to rise globally as the world's population ages.

Pathological hallmarks of AD include neurofibrillary tangles (NFTs) and neurite plaques. NFTs result from abnormal hyperphosphorylation of Tau protein, which disrupts neuronal morphology and impairs short-term and long-term synaptic plasticity (Naseri et al. 2019). Neurite plaques are aggregates of amyloid beta (Aβ) peptides composed of 40 (Aβ40) or 42 (Aβ42) amino acids. Aβ42 is especially prone to aggregation due to its higher rate of fibrilization and insolubility (Lane et al. 2018). Elevated brain levels of Aβ42 can cause neurotoxicity and neuron death as evidenced by impaired learning and recognition memory (Jaeger et al. 2009). Additionally, formation of Aβ plaques triggers microglia to polarize from a quiescent state to an activated phenotype. Microglia are myeloid cells that can perform proinflammatory/pro-killing or immunoregulatory functions in the brain (Ronaldson & Davis, 2020). They are also capable of phagocytosis, which contributes to the ability of microglia to remove cellular debris and contribute to neural repair (Akhmetzyanova et al. 2019; Ronaldson & Davis, 2020). These pathological processes suggest an opportunity for therapeutic targeting of neurons and microglia to promote neuroprotection in the setting of AD.

Efforts to develop disease-modifying AD drugs have so far overwhelmingly resulted in failure, and past developments have had little success in improving cognitive and functional ability in individuals affected by AD. Among these developments are large molecule therapeutics such as monoclonal antibodies and small molecules such as γ-secretase inhibitors and β-site amyloid precursor protein cleaving enzyme (BACE) inhibitors. In preclinical trials, the monoclonal antibody bapineuzumab was shown to lower the amount of Aβ present in the brain of mice, thus reducing AD pathology (Bard et al., 2000). Despite encouraging results, the drug failed to show significant improvement in functional or cognitive ability in treated patients in a phase three trial (Salloway et al., 2014). The drug also showed no significant difference in amyloid load or CSF phosphorylated tau levels between treatment and placebo groups in another phase three clinical trial (Vandenberghe et al., 2016). Other monoclonal antibodies that showed promising results but ultimately resulted in failure are solanezumab and gantenerumab. Another class of therapeutic developments currently being tested for treatment of AD are γ-secretase inhibitors. Despite promising results in early trials, phase three clinical trials for the γ-secretase inhibitor semagacestat were halted because of lack of efficacy and a worsening of function and cognition in patients (Doody et al., 2013). Another failed drug aimed at AD in this class is avagacestat. Other class of failed therapeutics include BACE inhibitors. In early studies, the BACE inhibitor atabecestat was shown to decrease CSF Aβ levels in individuals with early-stage AD (Timmers et al., 2018). However, results from phase two clinical trials and a long-term safety and tolerability study showed a trend of worsening cognitive assessment scores in treated patients (Novak et al., 2020). Verubecestat is another BACE inhibitor that was shown to decrease CSF Aβ levels in patients with AD (Kennedy et al., 2016) but was ultimately terminated because of failure in several later trials (Egan et al., 2019a; Egan et al., 2019b).

The multiple failures of therapeutics in clinical trials highlight a fundamental flaw of single-drug for single-target hypothesis for AD drug discovery and suggests a critical need to target multiple pathways simultaneously for a complex disease. Experiments conducted during the course of developing embodiments for the present invention resulted in the development of an integrative systems biology approach (Petyuk et al. 2018) to construct data-driven causal probabilistic models of gene and protein regulatory networks. These network models provided a systematic view of the molecular pathways and mechanisms to identify potential upstream master regulator genes and/or proteins (collectively named key drivers or therapeutic targets) that potentially modulate AD pathology. Based on the de-novo network-derived targets for AD, the inventors further developed a compound screening and prioritization process that repurposed FDA approved, natural and investigational compounds with known on-targets in the DrugBank against these therapeutic targets. The inventors identified three natural compounds (i.e., oxidized and reduced forms of nicotinamide adenine dinucleotide (NAD+, NADH) and phenethyl isothiocyanate (PEITC)) regulating total 97 AD key drivers and 212 significant (FDR<0.05) pathways identified by computational network analysis. The inventors then validated therapeutic effects of these individual compounds and their combinations in in vitro cell-based assays and two in vivo AD mice models (3xTg and hTau). The in vitro assays assessing neuroprotection and microglial phagocytosis demonstrated NAD+, NADH, PEITC individually and in combination significantly promoted neuron survival under AD condition and stimulated microglial anti-inflammatory phagocytosis of neurotoxic Aβ42. In addition, these compounds both prevented and rescue neural injury. The 3xTg AD mice treated with NAD+, NADH, PEITC individually showed significant reduction in the levels of neurotoxic Aβ42 and ratio of Aβ42 to Aβ40. The humanized Tau mice treated with PEITC showed significant reduction in the level of hyperphosphorylated Tau (p231Tau) and ratio of p231Tau to Tau. The brain proteomic data generated from 3xTg AD mice further confirmed NAD+ and NADH significantly perturbed pathways related to mitochondrial metabolism and energy production, and PEITC significantly modulated pathways involved in immune responses, and phagocytosis, which validated the computational analysis. All the results point towards the effectiveness of these natural products as AD therapeutics.

Accordingly, the present invention relates generally to neurodegenerative diseases and conditions (e.g., Alzheimer's disease) characterized with dysfunctional energetic function, unregulated microglia phagocytic activity and other related de-regulated biological functions. This invention further relates to methods and compositions for treating such neurodegenerative diseases and conditions with pharmaceutical compositions comprising one or more of phenyl isothiocyanate (PEITC), an analog of PEITC, oxidized nicotinamide adenine dinucleotide (NAD+), reduced nicotinamide adenine dinucleotide (NADH), nicotinamide mononucleotide (NMN), nicotinamide riboside (NR), a chemical moiety comprising isothiocyanate (e.g., 7-methylsulfinylheptyl, 8-methylsulfinyloctyl), vitamin c (ascorbate acid), glutathione, and myrosinase, wherein such compositions are capable of protecting neurons from cell death and unregulated microglia phagocytic activity.

In certain embodiments, the present invention provides a method of treating a mammal suffering from a condition characterized with dysfunctional energetic function, unregulated microglia phagocytic activity, and other related de-regulated biological functions comprising administering to the mammal a composition comprising one or more agents capable of protecting neurons from cell death and unregulated microglia phagocytic activity.

In certain embodiments, the present invention provides a method for preventing and/or inhibiting neuronal cell death in a mammal in need thereof, the method comprising administering to the mammal a composition comprising one or more agents capable of protecting neurons from cell death and unregulated microglia phagocytic activity.

In certain embodiments, the present invention provides a method for preventing and/or inhibiting unregulated microglia phagocytic activity in a mammal in need thereof, the method comprising administering to the mammal a composition comprising one or more agents capable of protecting neurons from cell death and unregulated microglia phagocytic activity.

In some embodiments, the condition characterized with dysfunctional energetic function, unregulated microglia phagocytic activity, and other related de-regulated biological functions is one or more of a neurodegenerative disorder, aging, systemic inflammation, neuroinflammation, cancer, and diabetes.

In some embodiments, the neurodegenerative disorder selected from AD, Parkinson's disease, Huntington's disease, amyotrophic lateral sclerosis, motor neuron disease, subjective memory complaints, and mild cognitive impairment (MCI). In some embodiments, the AD is an early stage, prodromal phase of AD or late stage.

In some embodiments, the mammal is a human patient.

In certain embodiments, the present invention provides a method for preventing and/or inhibiting neuronal cell death in a subject suffering from a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) comprising, consisting of, or consisting essentially of administering to the subject a therapeutically effective amount of one or more agents capable of preventing and/or inhibiting unregulated microglia phagocytic activity.

In certain embodiments, the present invention provides for preventing and/or inhibiting unrelated microglia phagocytic activity in neuronal cells of a subject suffering from a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) comprising, consisting of, or consisting essentially of administering to the subject a therapeutically effective amount of one or more agents capable of preventing and/or inhibiting unregulated microglia phagocytic activity.

In certain embodiments, the present invention provides a method of preventing the onset of a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) in a subject (e.g., a human subject) comprising, consisting of, or consisting essentially of administering to the subject a therapeutically effective amount of one or more agents capable of preventing and/or inhibiting unregulated microglia phagocytic activity.

In certain embodiments, the present invention provides a method of treating and/or ameliorating the symptoms of a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) in a subject (e.g., a human subject) comprising, consisting of, or consisting essentially of administering to the subject a therapeutically effective amount of one or more agents capable of preventing and/or inhibiting unregulated microglia phagocytic activity.

The compositions, methods, systems, and kits described herein are not limited to a specific type or kind of agent capable of protecting neurons from cell death and unregulated microglia phagocytic activity. In some embodiments, the agent is selected from PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the agent is a pharmaceutical composition comprising a therapeutically effective amount of one or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase.

The compositions, methods, systems, and kits described herein are not limited to a specific type or kind of PEITC analog.

In some embodiments, a PEITC analog is any chemical moiety related to watercress and/or other cruciferous plant extraction and the structures related to PEITC.

In some embodiments, the PEITC analog is selected from

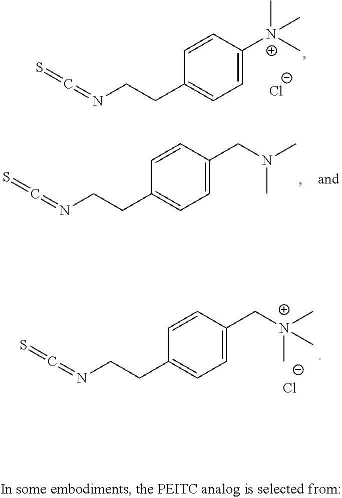 [SMILES: C[N+](C)(C)c1ccc(CCN=C=S)cc1 ; [Cl-] ; CN(C)Cc1ccc(CCN=C=S)cc1 ; C[N+](C)(C)Cc1ccc(CCN=C=S)cc1 ; [Cl-]]

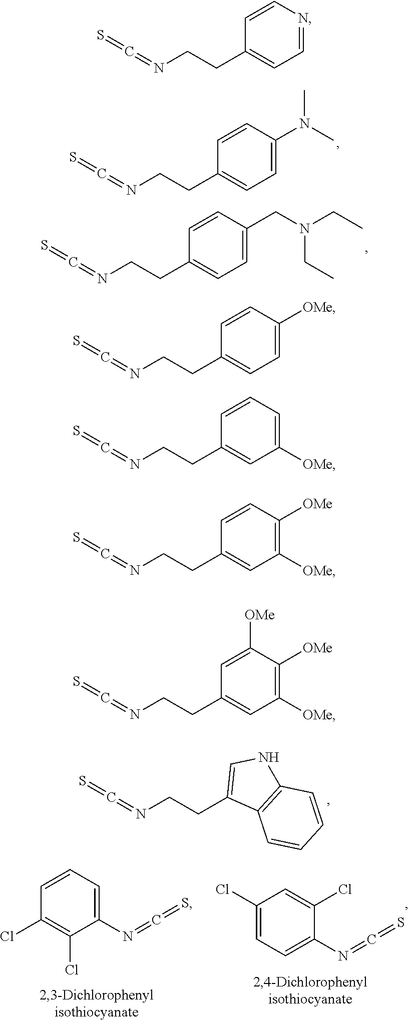

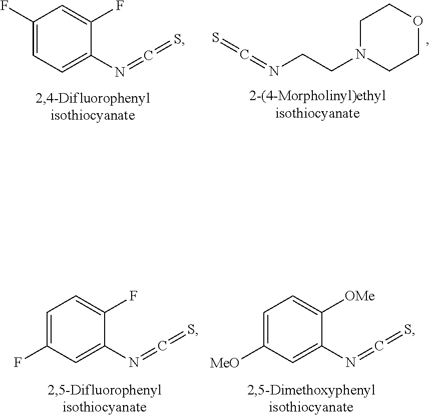

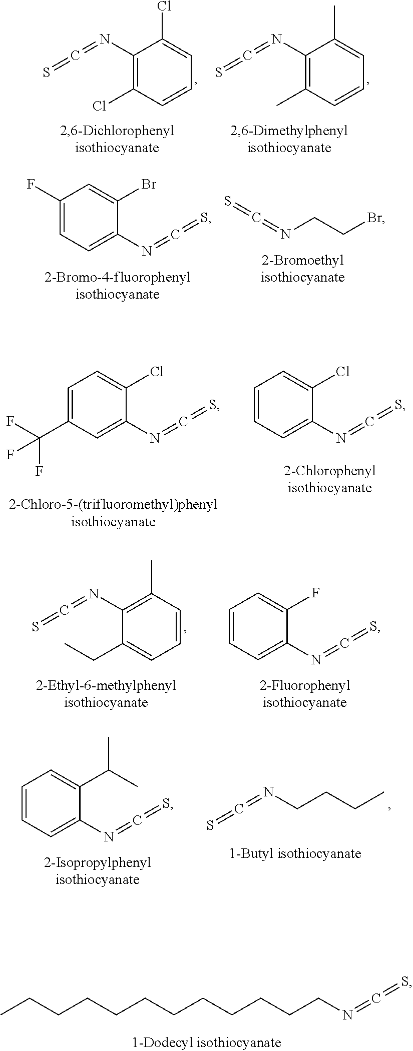

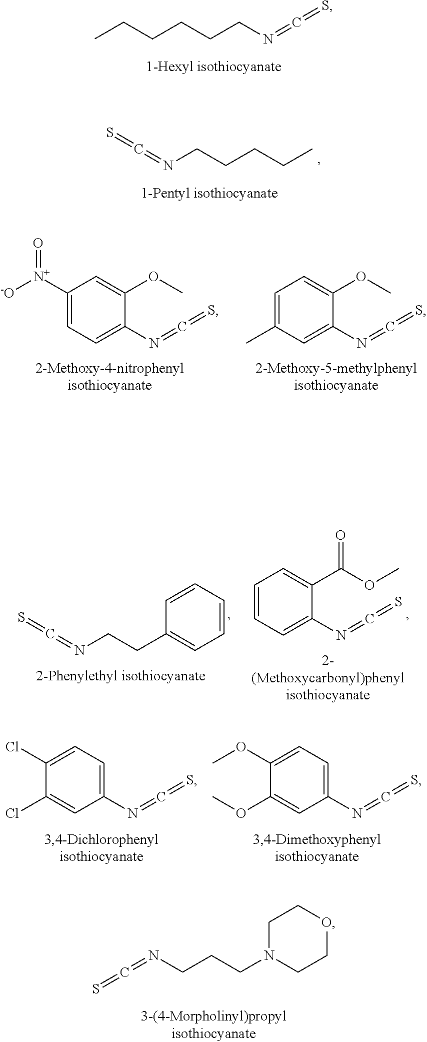

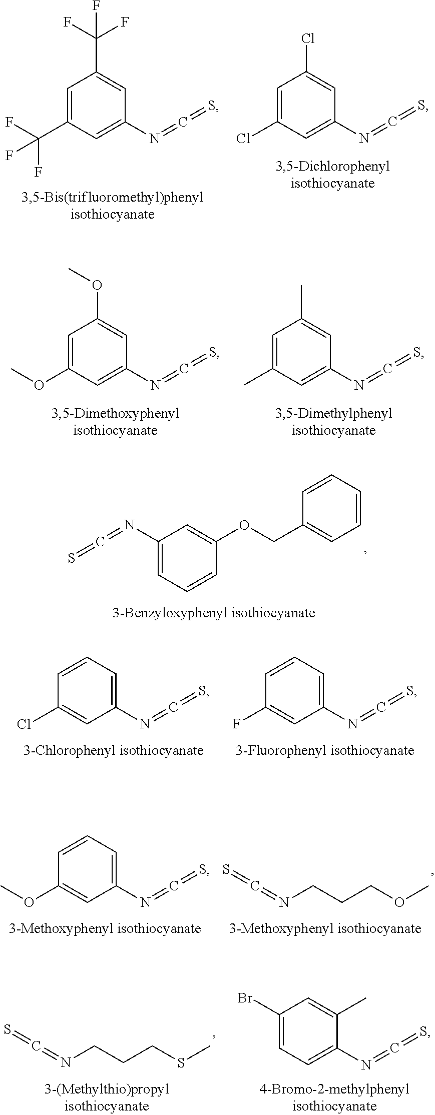

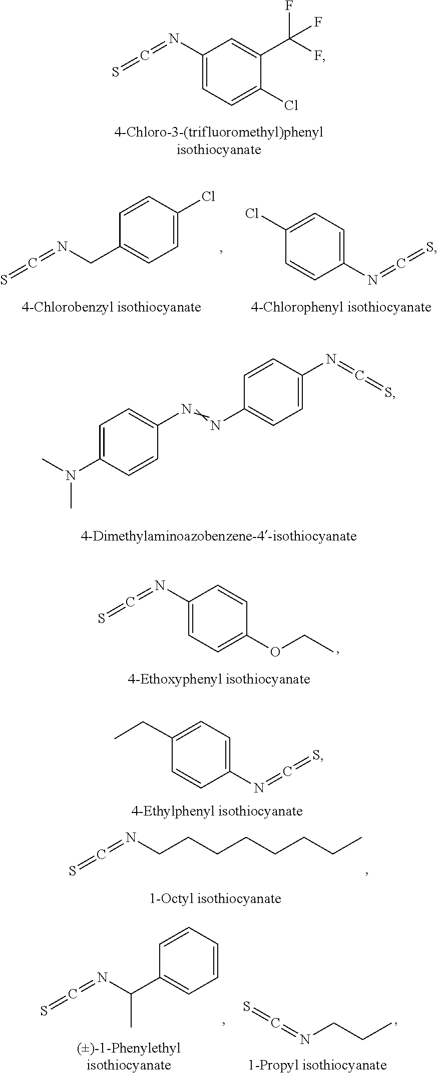

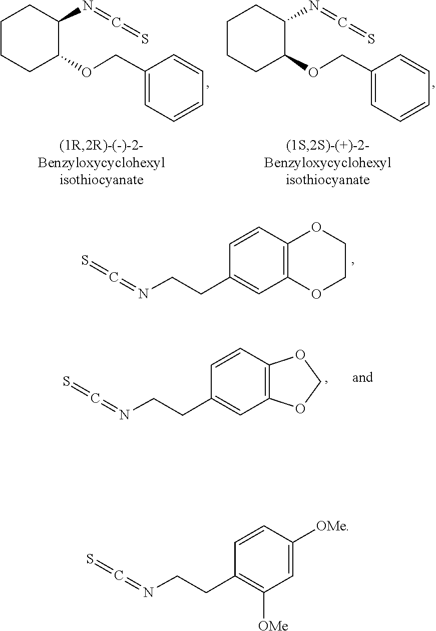

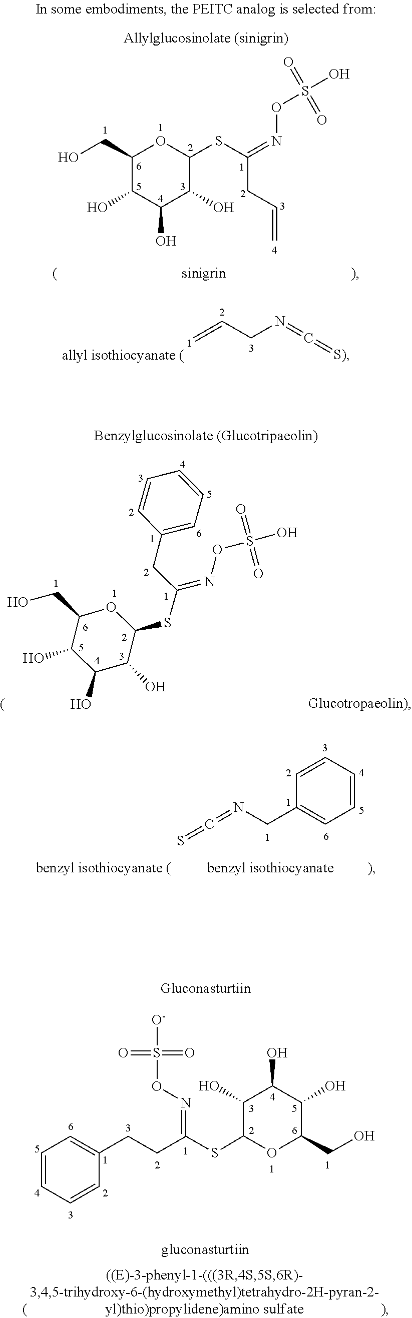

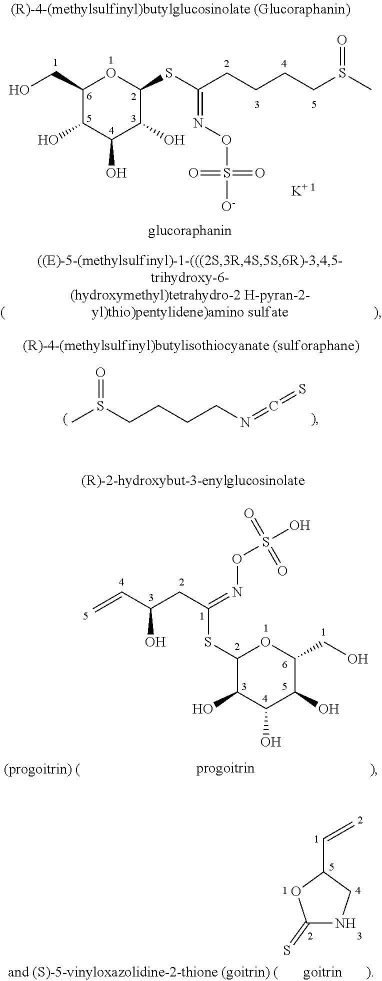

In some embodiments, the agent capable of protecting neurons from cell death and unregulated microglia phagocytic activity is derived from natural plants and seeds, and their extracts or derivatives. In some embodiments, the agent capable of protecting neurons from cell death and unregulated microglia phagocytic activity is derived from watercress, Cruciferous Vegetables, mustard, white mustard (Sinapis alba), garden cress (Lepidium sativum), wasabi (Wasabia japonica), and daikon (Raphanus sativus In some embodiments, the agent capable of protecting neurons from cell death and unregulated microglia phagocytic activity is derived from members of the family Brassicaceae, including yellow mustard (Brassica juncea), rape seed (Brassica napus). and common dietary Brassicas including, but not limited to, broccoli, cauliflower, cabbage, bok choy, kale, Papaya seeds, and cabbage aphid.

Myrosinase is a member of the glycoside hydrolase family. Myrosinase possesses several similarities with the more ubiquitous O-glycosidases, however, myrosinase is the only known enzyme found in nature that can cleave a thio-linked glucose. Its known biological function is to catalyze the hydrolysis of a class of compounds called glucosinolates.

Isothiocyanates (ITCs) are formed by the breakdown of glucosinolates, which are major constituents of cruciferous vegetables (watercress, cabbage, brussels sprouts, cauliflower, etc.). The enzyme myrosinase in plants and the microflora in the gastrointestinal tract are responsible for the release of ITCs from glucosinolates after physical damage to cruciferous vegetables (harvesting, cutting, or chewing) and after dietary ingestion, respectively. In humans, consumption of dietary glucosinolates is estimated to be about 300 mg/day from various cruciferous vegetables and, for every 56.8 g of watercress consumed, approximately 12 mg of PEITC is released.

A significant number of studies have shown a positive pharmacokinetic profile for orally administered PEITC. PEITC is highly bioavailable after oral administration. A single dose of 10-100 μmol/kg PEITC in rats resulted in bioavailability ranging between 90-114%. The high bioavailability was accompanied by low clearance as well as high protein binding. Increased bioavailability of PEITC was also observed in another study with repeated dosing of PEITC. Furthermore, about 928.5±250 nM peak plasma concentration of PEITC was achieved in human subjects, after the consumption of 100 g watercress.

The hydrolysis of glucosinolates, which is catalyzed by a class of enzymes called myrosinases (β-thioglucosidases), leads to the formation of breakdown compounds, such as thiocyanates, isothiocyanates, indoles, oxazolidine-2-thiones (e.g., goitrin), epithionitrile, and nitrile. In intact plant cells, myrosinase is physically separated from glucosinolates. Yet, when plant cells are damaged, myrosinase is released and comes in contact with glucosinolates, catalyzing their conversion into highly reactive metabolites that impart a pungent aroma and spicy (some say bitter) taste. Likewise, when raw cruciferous vegetables are chopped during the food preparation process, glucosinolates are rapidly hydrolyzed by myrosinase, generating metabolites that are then absorbed in the proximal intestine. In contrast, cooking cruciferous vegetables before consumption inactivates myrosinase, thus preventing the breakdown of glucosinolates. However, lightly cooking (i.e., light steam for <5 minutes) will preserve some of the myrosinase and allow for isothiocyanate conversion. A small fraction of intact glucosinolates may be absorbed in the small intestine, but a large proportion reaches the colon. In the colon, myrosinase produced by the microbiota can catalyze the generation of a wide range of metabolites from glucosinolates, depending on the pH and the presence of cofactors. Once absorbed, glucosinolate-derived isothiocyanates (like sulforaphane) are promptly conjugated to glutathione by a class of phase II detoxification enzymes known as glutathione S-transferases (GSTs) in the liver, and then sequentially metabolized in the mercapturic acid pathway. This mechanism is meant to increase the solubility of isothiocyanates, thereby promoting a rapid excretion in the urine. Using sulforaphane as the model isothiocyanate, it has indeed been established that its metabolites—sulforaphane-glutathione, sulforaphane-cysteine-glycine, sulforaphane-cysteine, and sulforaphane N-acetylcysteine—collectively known as dithiocarbamates, are ultimately excreted in the urine.

The composition and content of glucosinolates in cruciferous vegetables are relatively stable but depend on the genus and species and can vary with plant growing and post-harvest storage conditions and culinary processing. Since most cruciferous vegetables are cooked prior to eating, bacterial myrosinase in the gut, rather than plant myrosinase, is responsible for the initial step in glucosinolate degradation. In a feeding study involving 45 healthy subjects, the mean conversion rate of glucosinolates (of which 85% was glucoraphanin) to dithiocarbamates over a 24-hour period was estimated to be around 12% with wide variations among participants (range, 1.1 to 40.7%). In contrast, 70%-75% of ingested isothiocyanates were found to be metabolized to dithiocarbamates. Therefore, following the ingestion of cooked cruciferous vegetables, the conversion of glucosinolates into isothiocyanates by gut bacteria appears to be a limiting step in the generation of dithiocarbamates. However, differences in individuals' capacity to metabolize glucosinolates have not been linked to differences in gut microbiota composition.

Ascorbate (Vitamin C) is a known cofactor of myrosinase, serving as a base catalyst in glucosinolate hydrolysis. For example, myrosinase isolated from daikon (Raphanus sativus) demonstrated an increase in V max from 2.06 μmol/min per mg of protein to 280 μmol/min per mg of protein on the substrate, allyl glucosinolate (sinigrin) when in the presence of 500 μM ascorbate. Sulfate, a byproduct of glucosinolate hydrolysis, has been identified as a competitive inhibitor of myrosinase. In addition, 2-F-2-deoxybenzylglucosinolate, which was synthesized specifically to study the mechanism of myrosinase, inhibits the enzyme by trapping one of the glutamic acid residues in the active site, Glu 409.

Plants known to have evolved a myrosinase-glucosinolate defense system include: white mustard (Sinapis alba), garden cress (Lepidium sativum), wasabi (Wasabia japonica), and daikon (Raphanus sativus), as well as several members of the family Brassicaceae, including yellow mustard (Brassica juncea), rape seed (Brassica napus), and common dietary Brassicas like broccoli, cauliflower, cabbage, bok choy, and kale. The bitter aftertaste of many of these vegetables can often be attributed to the hydrolysis of glucosinolates upon tissue damage during food preparation or when consuming these vegetables raw. Papaya seeds use this method of defense, but not the fruit pulp itself.

Myrosinase has also been isolated from the cabbage aphid. This suggests coevolution of the cabbage aphid with its main food source. The aphid employs a similar defense strategy to plants. Like its main food source, the cabbage aphid compartmentalizes its native myrosinase and the glucosinolates it ingests. When the cabbage aphid is attacked and its tissues are damaged, its stored glucosinolates are activated, producing isothiocyanates and deterring predators from attacking other aphids.

In certain embodiments, the one or more agents capable of protecting neurons from cell death and unregulated microglia phagocytic activity may be comprised within any type or kind of composition. For example, in some embodiments, such a composition may be an over-the-counter composition, a pharmaceutical composition, or any kind of cosmetic composition.

In certain embodiments, the present provides the following compounds:

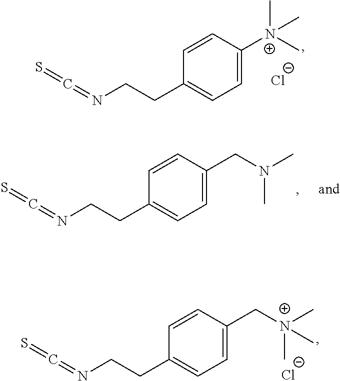 [SMILES: C[N+](C)(C)c1ccc(CCN=C=S)cc1 ; [Cl-] ; CN(C)Cc1ccc(CCN=C=S)cc1 ; C[N+](C)(C)Cc1ccc(CCN=C=S)cc1 ; [Cl-]]

including pharmaceutically acceptable salts, solvates, and/or prodrugs thereof.

In certain embodiments, the present invention provides a composition comprising one or more of the following: PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises two or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises three or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition is an over-the-counter composition, or a pharmacological prescription.

In certain embodiments, the present invention provides an over-the-counter composition comprising one or more of the following: PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises two or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises three or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the composition comprises PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase. In some embodiments, the over-the-counter composition is a tablet, capsule, powder, suspension, or solution.

The methods and compositions of the present invention are useful in treating mammals. Such mammals include humans as well as non-human mammals. Non-human mammals include, for example, companion animals such as dogs and cats, agricultural animals such live stock including cows, horses and the like, and exotic animals, such as zoo animals.

Treatment can include administration of an effective amount of one or more of an agent capable of protecting neurons from cell death and unregulated microglia phagocytic activity (e.g., one or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase).

Administration can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, oral, otic, biliary perfusion, cardiac perfusion, priodontal, rectal, spinal subcutaneous, sublingual, topical, intravaginal, transermal, ureteral, or urethral. Dosage forms can be aerosol including metered aerosol, chewable bar, capsule, capsule containing coated pellets, capsule containing delayed release pellets, capsule containing extended release pellets, concentrate, cream, augmented cream, suppository cream, disc, dressing, elixer, emulsion, enema, extended release fiber, extended release film, gas, gel, metered gel, granule, delayed release granule, effervescent granule, chewing gum, implant, inhalant, injectable, injectable lipid complex, injectable liposomes, insert, extended release insert, intrauterine device, jelly, liquid, extended release liquid, lotion, augmented lotion, shampoo lotion, oil, ointment, augmented ointment, paste, pastille, pellet, powder, extended release powder, metered powder, ring, shampoo, soap solution, solution for slush, solution/drops, concentrate solution, gel forming solution/drops, sponge, spray, metered spray, suppository, suspension, suspension/drops, extended release suspension, swab, syrup, tablet, chewable tablet, tablet containing coated particles, delayed release tablet, dispersible tablet, effervescent tablet, extended release tablet, orally disintegrating tablet, tampon, tape or troche/lozenge.

Intraocular administration can include administration by injection including intravitreal injection, by eyedrops and by trans-scleral delivery.

Administration can also be by inclusion in the diet of the mammal such as in a functional food for humans or companion animals.

It is also contemplated that certain formulations containing the compositions (e.g., compositions comprising one or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase) capable of protecting neurons from cell death and unregulated microglia phagocytic activity are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methylcellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated such as to provide rapid, sustained, or delayed release of the active ingredients after administration to the patient by employing procedures well known in the art. The formulations can also contain substances that diminish proteolytic degradation and promote absorption such as, for example, surface-active agents.

The specific dose can be calculated according to the approximate body weight or body surface area of the patient or the volume of body space to be occupied. The dose will also depend upon the particular route of administration selected. Further refinement of the calculations necessary to determine the appropriate dosage for treatment is routinely made by those of ordinary skill in the art. Such calculations can be made without undue experimentation by one skilled in the art in light of the activity in assay preparations such as has been described elsewhere for certain compounds (see for example, Howitz et al., Nature 425:191-196, 2003). Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

The present invention also provides kits comprising one or more of agents capable of protecting neurons from cell death and unregulated microglia phagocytic activity (e.g., one or more of PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase) and instructions for administering the agent to an animal (e.g., a human patient suffering from a neurodegenerative disorder (e.g., AD)). The kits may optionally contain other therapeutic agents.

### EXPERIMENTAL

The following examples are provided to demonstrate and further illustrate certain preferred embodiments of the present invention and are not to be construed as limiting the scope thereof. As used herein, the use of pronouns (e.g., “our”, “we”, etc.) refers to the inventors.

### Example I

### The AD Population and Data Pre-Processing

A translational discovery pipeline for drug discovery starts with integrative analysis of multi-layer omics data (genotyping, RNA-seq, proteomics) generated from three human cohorts sponsored by the Accelerating Medicines Partnership-Alzheimer's Disease (AMP-AD) consortium including i) the post-mortem dorsolateral prefrontal cortex (DLPFC) of 612 persons in the Religious Order Study and the Memory and Aging Project (ROSMAP) (Bennett et al., 2012b, 2012a; Jager et al., 2018), ii) the prefrontal cortex of 306 subjects in the Mount Sinai Brain Bank (MSBB) RNA-seq study (Wang et al., 2018), and iii) temporal cortex (TCX) of 266 patients in the Mayo RNA-seq study (Allen et al., 2016). In addition to AMP-AD data, we (the inventors) included the human BRAINOME dataset (RuiChangBrain2018) containing i) frontal and temporal cortical regions of 345 subjects in KRNONOSII cohorts and ii) frontal cortical regions of 409 subjects in RUSH cohort. For RNA-seq data pre-processing, we applied a consensus data processing pipeline including voom normalization, variance partition analysis (VPA) and covariate adjustment. For proteomics data pre-processing, we applied normalization, variance partition analysis and covariate adjustment. For genotyping data pre-processing, we performed imputation.

Besides brain region RNA-seq data, as brain tissue consists of various cell types, including neurons, endothelial and glial cells, we applied the computational framework population-specific expression analysis (PSEA)[23] to deconvolve bulk-tissue (brain region) RNA-seq data from post-mortem brain regions and isolate a neuron-specific and microglial-specific gene expression signal. We chose the PSEA method over other popular methods, such as Cibersort [24], dtangle [25], DSA [26], or NNLS [27], because these methods cannot directly estimate cell-type specific residuals from the bulk-tissue RNA-seq data, instead only estimating cell fraction in a bulk-tissue sample. We demonstrated the robustness of this deconvolution method using random selection of neuronal biomarkers derived from single-cell RNA-seq (scRNAseq) studies [28-32]. Next, we applied a novel systems biology approach to integrate whole-genome genotype data with the brain regionl and single cell-type RNAseq, and to build causal network models of AD.

### Translational Computational Systems Biology Approach to Repurpose Compounds for AD

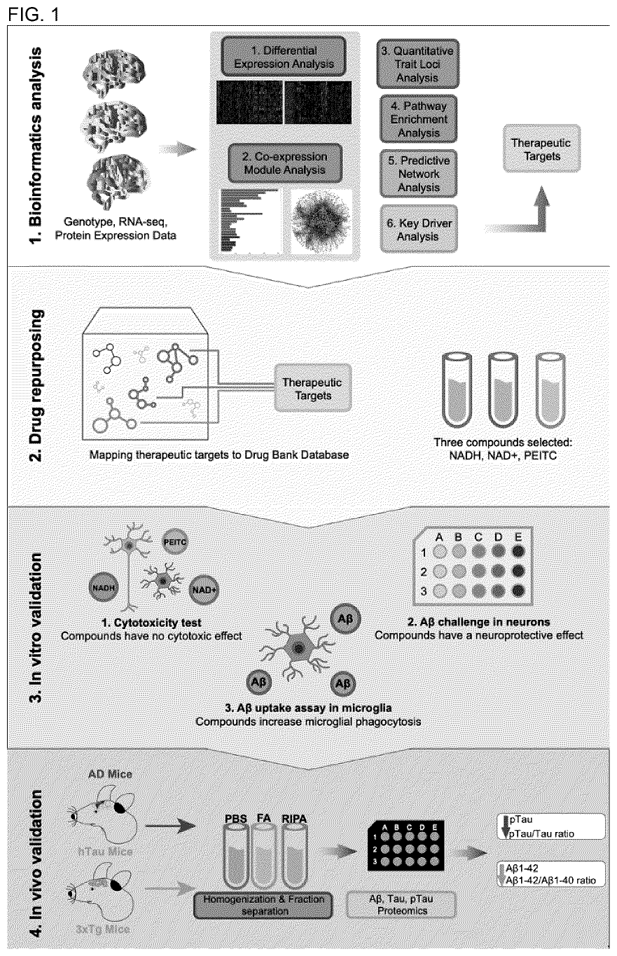

The computational drug repurposing pipeline (FIG. 1) is centered on a cutting-edge computational systems biology model which integrates the multi-omics (genotyping, RNA-seq, proteomics) data of matched subjects in a cohort to construct network models and to identify therapeutic targets. For every cell type/brain region in each cohort, we identified AD-related (gene and protein) expression signature, single-nucleotide variants associated with gene expression and protein level, transcriptomic and proteomic co-expression modules associated with AD, data-driven systems biology network model and AD-associated pathways, and a set of potential therapeutic targets for AD (Online Method).

To evaluate robustness of AD-related analysis results across different datasets, we performed a meta-analysis of the above results across all cell types/brain regions, and cohorts. After identified the robust therapeutic targets, we repurposed both FDA approved and investigational compounds by matching the list of targets to known targets of compounds in DrugBank (version 5.0), which contains 9591 drug entries including 2037 FDA-approved small molecule drugs, 241 FDA-approved biotech (protein/peptide) drugs, 96 nutraceuticals and over 6000 experimental drugs. The repositioned compounds are then ranked according to the number of predicted AD targets a compound hit. Consequently, we prioritized three repurposed compounds which are validated to have neuroprotective effects on human primary neurons and promoting amyloid-beta uptake in human primary microglial cells under AD condition.

### Meta-Transcriptomic Analysis Revealed Robust Differential Expression Signature in Alzheimer's Disease

To identify robust expression signature significantly associated with AD, we performed dataset-specific differential expression (DE) analysis for all brain regions, cell types and cohorts. By comparing expression values from AD and pathologically confirmed controls (CN), we identified different number of significant (FDR<0.05) DE genes and proteins between AD and CN samples in each dataset.

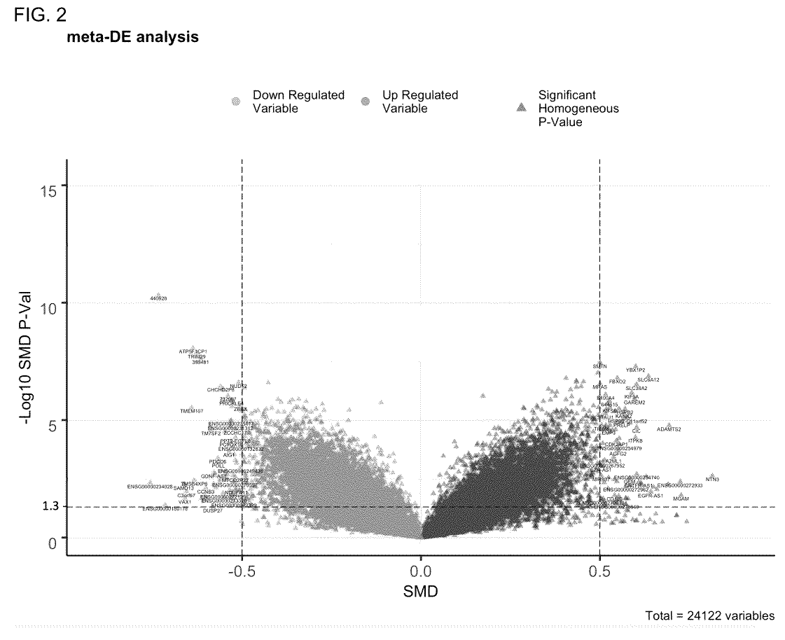

To examine the robustness of DE signature, we performed a meta-analysis on the differential expression for total 19,119 variables (genes and proteins) across all datasets. We calculated the overall standardized mean difference (SMD) and its p-value for every variable with a random-effect model. A positive SMD value indicates a variable is overall up-regulated in AD across all datasets and a negative SMD value indicates a variable is overall down-regulated in AD across all datasets. Among the 19,119 variables, there are 4,463 significant up-regulated genes/proteins (SMD>0 and SMD's p-value<0.05) and 3,796 significant down-regulated genes/proteins (SMD<0 and SMD's p-value<0.05) across all the datasets. We further calculated p-value of heterogeneity for every variable across all datasets. A variable's expression pattern is considered homogeneous/robust across all datasets if its heterogeneity p-value is larger than 0.05. As a result, we identified 3,344 variables which are significantly and robustly up-regulated in AD and 2,939 variables which are significantly and robustly down-regulated in AD. We highlighted the most robust and significantly up- and down-regulated genes/proteins associated with AD in FIG. 2.

To evaluate the quality of DE signature derived from individual dataset, we calculated overlap significance between individual significant (FDR<0.05) DE signature with the significant (p-value<0.05) meta-DE signature. Out of the 10 individual DE lists, all 8 DE (gene) signatures are significantly overlapping with the meta-DE signature whereas none of the DE (protein) signature significantly overlapping with the meta-DE protein signature. The results indicated that meta-DE signature is a core AD-associated signature associated with AD across cohorts, brain regions, cell types and technical variance. In addition, we also identified individual DE signature sharing the common Meta-DE but also with unique signature specific to the cohort, brain region, and cell types. To retain the unique signature, we will proceed analysis with individual DE signature and residuals and integrate the therapeutic targets at last.

### Robust Genetic Variants Associated With AD Expression Traits Across AMP-AD

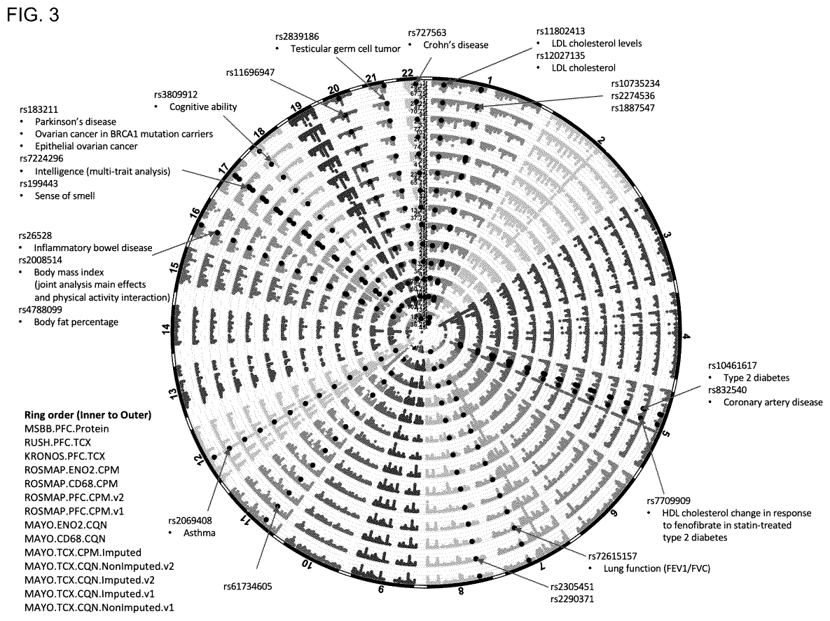

We analyzed association of gene and peptide expression traits with genome-wide variants assayed in every dataset we collected. For genotype variants assayed by SNP arrays in ROSMAP and MAYO cohorts, we performed imputation (Online Methods). Overall, we tested allelic loci-transcript and allelic loci-peptide correlation across brain regions/cell types and cohorts which is summarized in FIG. 3. We found that 5609, 9041, 4343, 3089, 5813, 3875, 3331, 6103, 2563, 5186, 5059, 4200, 5460 genes tested were significantly correlated with allele dosage (FDR<0.05 or FDR<0.01) in the respective dataset: MAYO TCX CQN NonImputed v1, MAYO TCX CQN Imputed v1, MAYO TCX CQN Imputed v2, MAYO TCX CQN NonImputed v2, MAYO TCX CPM Imputed, MAYO CD68 CQN, MAYO ENO2 CQN, ROSMAP PFC CPM v1, ROSMAP PFC CPM v2, ROSMAP CD68 CPM, ROSMAP ENO2 CPM, KRONOS PFC TCX, RUSH PFC TCX, as well as 226 peptides tested were significantly corrected with allele dosage (FDR<0.05) in MSBB PFC Protein. Of these cis expression quantitative trait loci (cis-eQTL) detected in each dataset, 7 cis-eQTL were overlapping across all 14 datasets and 2093 cis-eQTL were overlapping across 13 out of 14 datasets in FIG. 3 and 102,548 cis-eQTL were replicated in at least 50% of all datasets even though these datasets comprised different brain regions, cell types and cohorts. We examined the known association of total 829,970 significant cis-eQTLs across all datasets with diseases in GWAS catalog, which could help to identify co-morbid conditions with AD. We found that out of the 829,970 cis-eQTLs, 1,532 are significantly associated with at least one disease in GWAS catalog. Out of the 2093 robust cis-eQTLs, 16 are significantly associated with at least one disease in GWAS catalog (highlighted in FIG. 3 with their associated diseases).

### Identification of AD-Related Transcriptional Modules by Meta-Coexpression Module Analysis

While DE analysis revealed AD-related expression signature, the power of such analysis to detect a small-to-moderate expression difference is small. On the other hand, gene and protein expression traits from coherent biological pathways are correlated and clustered into modules of traits which can be recapitulated by constructing co-expression modules.

To identify co-regulated human transcriptomic and proteomic modules that were robustly observed in a generalized manner across all datasets generated from brain regions, cell types and cohorts, we firstly constructed a co-expression network of the AD samples in each dataset by using WGCNA (Parikshak et al., 2016) and identified total 253 dataset-specific co-expression modules (MAYO TCX RNA: 23; MAYO ENO2 RNA: 21; MAYO CD68 RNA: 46; ROSMAP PFC RNA: 27; ROSMAP ENO2 RNA: 15; ROSMAP CD68 RNA: 47; KRONOS PFC TCX RNA: 27; KRONOS PFC TCX Protein: 6; RUSH PFC TCX RNA: 29; RUSH PFC TCX Protein: 8).

To compare the structure of all co-expression modules, we calculated overlap enrichment of module-molecule membership between all pairs of modules across all datasets and identified 1690 module pairs with significant overlap (FDR<0.05 for Fisher's Exact Test). As expected, we found that co-expression modules from different datasets (regions, cell types, and cohorts) have good rate of replication indicating that there is no significant bias and/or batch effect in the processed residuals between the datasets.

Next, we characterized the functional relevance and AD-association of all 249 modules based on the following enrichment tests per module: 1) fold-enrichment for AD-related DE signature; 2) fold-enrichment for single cell-type biomarkers for single cell-type module network. In total, we selected 72 AD-associated modules (interchangeably AD-modules) that were significantly enriched for AD-related DE signature and single cell-type biomarkers. We evaluated the association significance with the AD pathological traits that is available in each dataset (MAYO: BRAAK, THAL; ROSMAP: BRAAK, CERAD, COGDX, KRONOS/RUSH: BRAAK, DET).

Last, we repeated the structural comparison among AD-associated modules by overlapping enrichment test and identified 430 significant module-pairs (FDR<0.05) out of all AD-module pairs across datasets. Similar to all modules, we found that AD-associated modules from different datasets (regions, cell types, and cohorts) have good rate of replication indicating that module selection is not biased by datasets.

In next section, we employed predictive network model to infer causality between the variables (genes and proteins) from AD-modules in each dataset.

### Data-Driven Causal Regulation Inference by Predictive Network for AD

The ultimate goal of the experiments described herein was to identify upstream pathways and robust master regulators (key drivers) of AD pathology for drug repurposing. As last step of the translational drug repurposing pipeline, we built a causal network of transcriptomic and proteomic regulations by integrating analysis from previous steps with RNA-seq and proteomic residuals for every dataset. In total, we built 78 predictive networks over different versions of cis-eQTLs, cell types (neuron/microglia), brain regions (TCX/PFC) and cohorts (ROSMAP/MAYO/MSBB).

To build the predictive network, we first pooled all variables of AD-associated modules in every dataset limiting the causal network model to AD pathology only. Noting that the data-driven approach may not fully capture known signaling pathways because the data is generated and analyzed from a specific biological layer at a time, we expanded the AD-associated seeding variables by integrating prior signaling pathways in the knowledge databases via PathFinder algorithm (BioRXiv, CSC, Brain) (Online Method). Third, we incorporated cis-eQTLs as structural prior in the network where cis-eQTLs causally affect the expression levels of associated variables as a source of systematic perturbation to infer causal relations.

To compare network models, we first extracted all (causal) edges from each network model. Similar to module meta-analysis, we calculated overlap of edges between all pairs of predictive network models and identified network pairs with significant overlap (FDR<0.05 for Fisher's Exact Test). As expected, most network structures that are significantly overlap with each other are within the same dataset, which reflects molecule and functional differences of regions and cell types related to AD pathology. Interestingly, we found that RNA network significantly overlaps with the multiscale (RNA-peptide) networks from KRONOS and RUSH cohorts, where these two multiscale networks significant overlap with each other confirming a replicated genetic regulatory mechanism associated with AD in the same brain region across the two cohorts. The two peptide networks from KRONOS and RUSH significantly overlap with each other indicating a robust PPI program associated with AD in the same brain regions across the two cohorts. The microglial and neuron-specific networks from ROSMAP significantly overlap with brain region network from ROSMAP indicating there might be a core subnetwork of pathways across neurons and microglia associated with AD pathology. To investigate the core mechanisms associated with AD across brain regions, cell types, and cohorts, we employed spine algorithm to derive a core subnetwork from each predictive network and evaluated the overlaps among these core structures. We found that the overlap significance among core subnetworks is consistent with the overlaps among entire predictive networks.

### Repurposing Therapeutic Drugs Targeting Multiple Pathological Pathways in AD

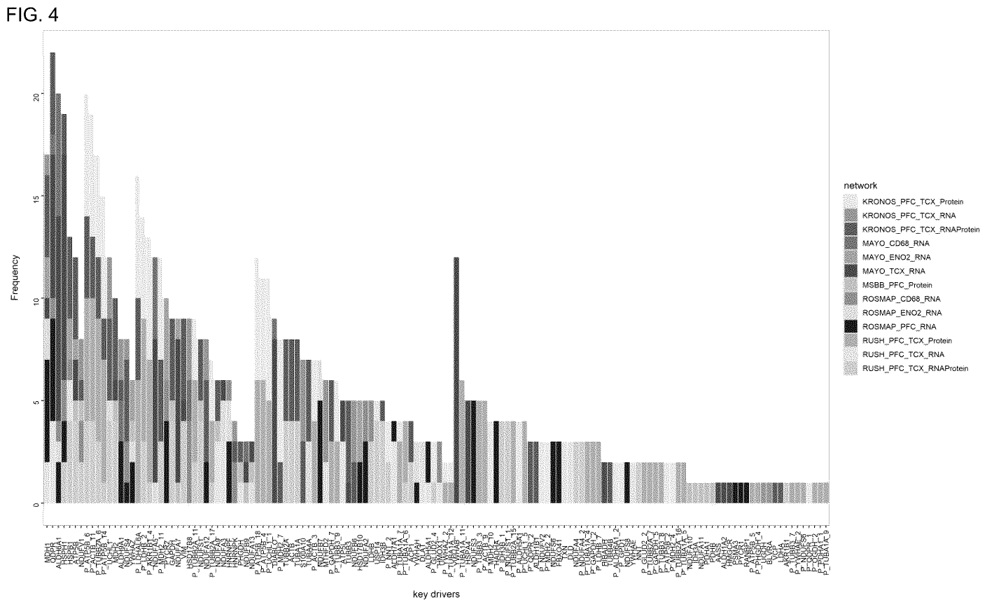

To identify potential therapeutic compounds for Alzheimer's disease, we firstly applied Key Driver Analysis (KDA) to the 78 predictive network models across brain regions, cell types and cohorts in the study. The KDA identified a list of key drivers/therapeutic targets in each network. Next, we repurposed total 13,682 drug entries in the latest DrugBank (version 5.1.7) including 2,646 approved small molecule drugs, 1,405 approved biologics (protein, peptide, vaccines, and allergenics), 131 nutraceuticals and over 6,402 experimental (discovery-phase) drugs, with additional 5,234 non-redundant protein (i.e. drug target, enzyme, transporter, carrier) sequences are linked to these drug entries, by matching the drugs with known targets to the key drivers derived identified from the 78 predictive network models. Based on the number of matched targets, we prioritized all 13,682 drugs compounds and selected three top compounds, i.e. NADH, NAD+, PEITC, hitting total 138 key drivers (genes and peptides) for AD in FIG. 4. After mapping peptides to gene, 138 key drivers are mapped to 85 unique genes.

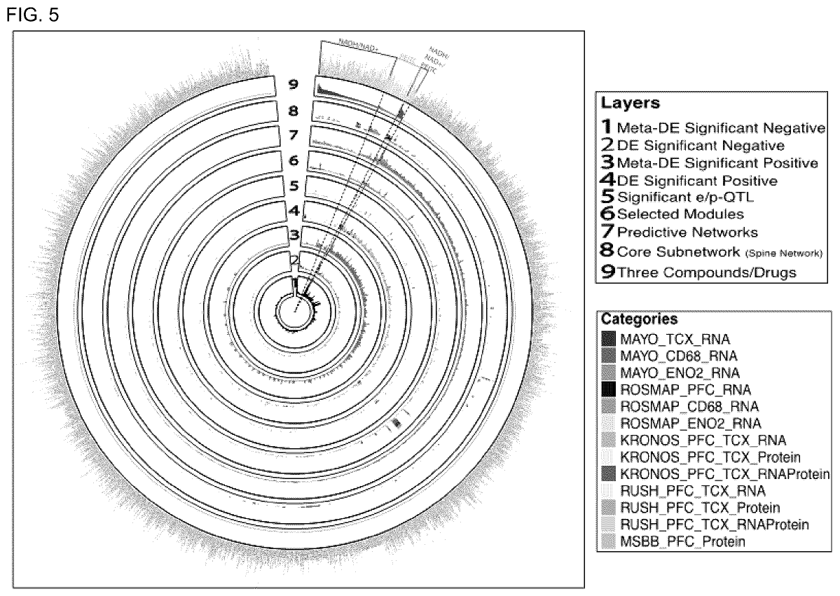

The translational drug-repurposing pipeline (FIG. 1) has generated an integrated landscape of AD-associated mechanisms by different analyses including AD-associated differential expression signature, genetic variants, co-expression modules, causal network models, therapeutic targets/key drivers and repositioned drugs. To gain therapeutic insights, we (the inventors) extracted significant pathways enriched by this set of AD-associated molecule landscape, including 10 AD-related up-/down-regulated DE signatures derived from individual dataset, 1 AD-related up-/down-regulated meta-DE signature, 14 lists of genes/peptides significantly associated with genetic variants in AD, 72 AD-associated modules, 78 AD-associated predictive networks, 78 AD-associated core subnetworks, and 1 set of 138 key drivers. Based on these 265 sets of AD-associated genomic phenotypes (see, FIG. 5), we identified total 2,776 non-repeating pathways that are significantly enriched by at least one set of phenotype. Interestingly, we found that NADH and NAD+ which hits the largest number of key drivers modulates mitochondrial-related pathways, such as TCA-cycle, respiratory electron transport, oxidative phosphorylation, glycolysis and gluconeogenesis, indicating that mitochondrial functions and mitochondrial energy metabolism is an important therapeutic target for Alzheimer's disease. Through the analysis, we nailed down 81 AD key driver genes and peptides in these pathways regulated by NADH and NAD+. In addition to NADH and NAD+, the third compound PEITC modulates chaperone-mediated protein folding pathways whose disruption is leading to accumulation of amyloid aggregates, and Tubulin folding pathway which forms the microtubules whose loss contributes to tau disassociation and abnormal tau accumulation in AD, parkin-ubiquitin in the proteasomal pathway. Parkin is E3-ubiquitin-protein ligase that ubiquitinates itself and specific substrate proteins playing a protective role by sequestering misfolded proteins and mutation in this gene will cause familial Parkison's disease(ref). Other important PEITC-regulated pathways related to inflammation, cell cycle, cell death, apoptosis and survival, such as TNF-alpha signaling, mTOR signaling pathway, P38 signaling mediated by MAPKAP kinase, activation of BAD and translocation to mitochondria, EGF/EGFR pathway, and FAS pathway. In addition, PEITC regulates phagosome functions of the cells, translocation of Glut4 (glucose transporter) to the cell membrane which is critical for glucose uptake from bloodstream by the cells, and energy homeostasis pathways, such as Liver kinase B1-mediated cellular events. PEITC also regulates cellular response to stress and DNA damage, such as regulation of HSF1-mediated heat shock response, activation of BH3-only proteins, and chromatine remoding pathways via regulating MTA family of proteins, whose functions are only recently recognized in the brain, eye, circadian rhythm, mammary gland biology, spermatogenesis, liver, immunomodulation and inflammation, cellular radio-sensitivity, and hematopoiesis and differentiation. One of the nervous pathways directly regulated by PEITC is Trk receptors signaling mediated by PI3K and PLC-gamma. Trk receptors are a family of tyrosine kinases that regulates synaptic strength and plasticity in the mammalian nervous system, which affect neuronal survival and differentiation through several signaling cascades. Through the analysis, 57 AD-driving genes and peptides (27 unique genes) in these pathways regulated by PEITC were nailed down.

### Cytotoxic Effect of NADH, NAD+ and PEITC on Primary Cultures of Human Neurons and Human Microglia

A defining characteristic of the many failed AD therapeutics has been the focus on targeting single biological pathways in an effort to achieve therapeutic efficacy. This is particularly apparent with drugs that were designed to specifically target Aβ plaques such as solanezumab (Doody et al. 2014) or avagacestat (Coric et al. 2015). This approach has identified three natural product compounds (i.e., NADH, NAD+, PEITC) that interact with molecular targets associated with mitochondrial bioenergetics and/or antioxidant effects, which indicate an ability to interact with multiple AD-associated pathways and/or multiple CNS cell types relevant to AD pathology. Furthermore, cellular concentrations of NADH and NAD+ have been shown to be decreased in patients with late onset AD (Sonntag et al. 2017). This fact further emphasizes that a key characteristic of the AD phenotype is impaired mitochondrial energy metabolism and an abnormal shift towards glycolytic energy production (Sonntag et al. 2017).

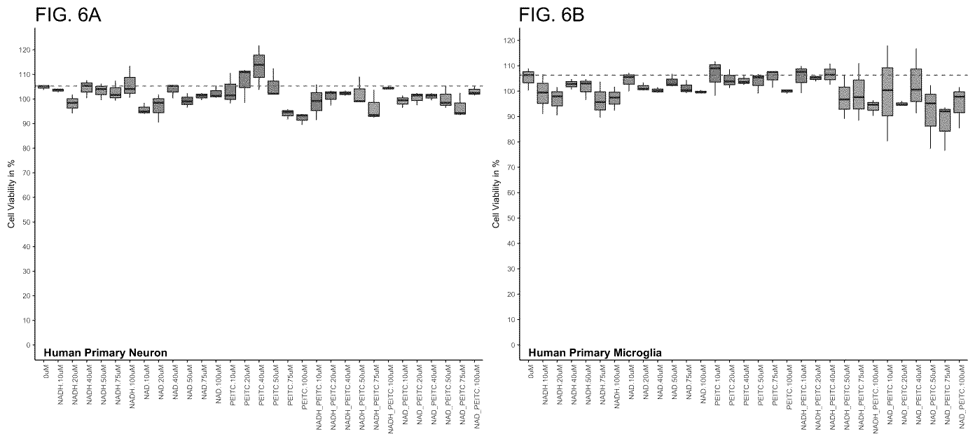

To evaluate therapeutic effects on AD of these three repositioned compounds discovered by a translational computational systems biology pipeline, we first validated the cytotoxic effect of each compound, administered individually or in combination, on primary cultures of human neurons and on primary cultures of human microglia. To perform these critical experiments, we incubated cultured cells with 0 μM (control condition), 10 μM, 20 μM, 40 μM, 50 μM, 75 μM, and 100 μM of each compound and two combinations (NAD+/PEITC and NADH/PEITC). After 24 h incubation at 37° C. in a humidified incubator, cell viability was measured using a 3-(4,5-dimethylthiazol-2-yl)-2,5-diphenyltetrazolium bromide (MTT) assay. Following treatment with NADH, NAD+, or PEITC and their combinations, cell viability in primary cultures of human neurons and microglia was not significantly altered as compared to control conditions (Dunnett's T3 adjusted p-value>0.05), suggesting that the three compounds, when administered individually, did not have a cytotoxic effect (FIG. 6A,B). Of particular importance to the therapeutic development of these compounds, combinations of NADH and PEITC or NAD+ and PEITC, when formulated in equimolar concentrations, did not significantly affect cell viability of both primary cultures of human neurons and primary cultures of human microglia (FIG. 6A,B). Overall, our results demonstrated that NADH, NAD+, PEITC individually or in combination are non-toxic to human CNS cells in vitro, which is a critical step in the preclinical validation of these compounds.

### Neuroprotective Effect of Natural Compounds on Primary Cultures of Human Neurons Exposed to Aβ1-42 Aggregates

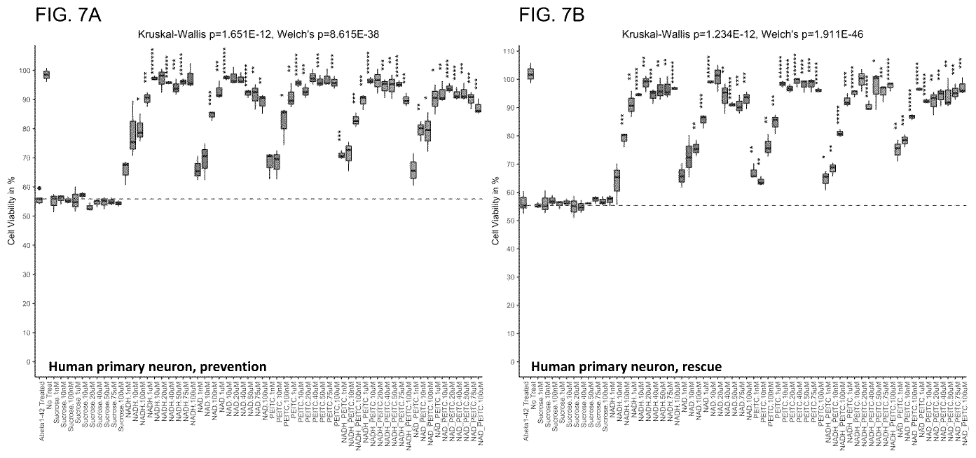

Next, we determined whether these compounds, administered independently or formulated in combination, could protect primary cultures of human neurons against neurotoxic effects of Aβ1-42 aggregates, an in vitro condition relevant to AD pathology. We evaluated both prevention and rescue of neuronal death by NADH, NAD+ and PEITC (FIG. 7). In both experiments, the cultured human neurons were exposed to 1 nM, 10 nM, 100 nM, 1 μM, 10 μM, 20 μM, 40 μM, 50 μM, 75 μM, and 100 μM of each compound, either individually or in equimolar combinations. As a baseline parameter associated with Aβ1-42 exposure, primary cultures of human neurons challenged by Aβ1-42 experienced a 50% or greater reduction in cell viability (FIG. 7). In the prevention experiment, cultures were exposed to fresh compounds every 24 h over the 48 h timeline of this experiment (i.e., 0 h, 24 h, and 48 h) to resemble a pharmacological regimen of once daily dosing. At 24 h, we added 1 μM of Aβ1-42 aggregates to the cell culture media and measured cell viability at 72 h using the MTT assay (Online Method, FIG. 7A). Our results showed that NADH, NAD+, PEITC either individually or in combination significantly prevented neuronal loss as compared to the negative control condition (i.e., cellular exposure to 1 μM Aβ1-42 without administration of any individual compounds or compound combinations). Additionally, we discovered that individual compounds have a wide effective dosage range from 100 nM to 100 μM in prevention of neuron loss following Aβ1-42 exposure. Interestingly and surprisingly, we discovered that equimolar combinations of NADH/PEITC and NAD+/PEITC exhibited synergistic effects of neuroprotection that expanded the minimal effective concentration to 1 nM and 10 nM respectively as compared to the 100 nM concentration for single compounds. To rule out false positive data points and experimental artifacts, we showed that sucrose did not prevent any neuronal death in response to Aβ1-42 aggregates (FIG. 7A). In the rescue experiments, primary cultures of human neurons were exposed to 1 μM of Aβ1-42 aggregates at 0 h for a period of 48 h to model existing cellular damage under conditions relevant to AD. After 24 h incubation with Aβ1-42, we added NADH, NAD+, PEITC, NADH/PEITC, and NAD+/PEITC directly to the culture media containing Aβ1-42 and incubated the cultured human neurons for an additional 24 h when cell viability was measured using our MTT assay. Similar to the prevention experiments, NADH and NAD+ significantly rescued neuronal death caused by Aβ1-42 with a wide dosage range from 100 nM to 100 μM, and PEITC significantly rescued the neuron death from 1 nM to 100 μM (FIG. 7B). The equimolar combination of NADH/PEITC and NAD+/PEITC exhibited synergistic neuroprotection with effective dosage ranges from 1 nM to 100 μM respectively. In summary, all five formulations of three repurposed natural compounds demonstrated significant effects in both preventing and rescuing neuronal loss caused by Aβ1-42 aggregate deposition, in vitro conditions that are characteristic of early-stage Alzheimer's disease.

### Phagocytosis-Promoting Effect of Natural Compounds in Primary Cultures of Human Microglia Exposed to Aβ1-42 Aggregates

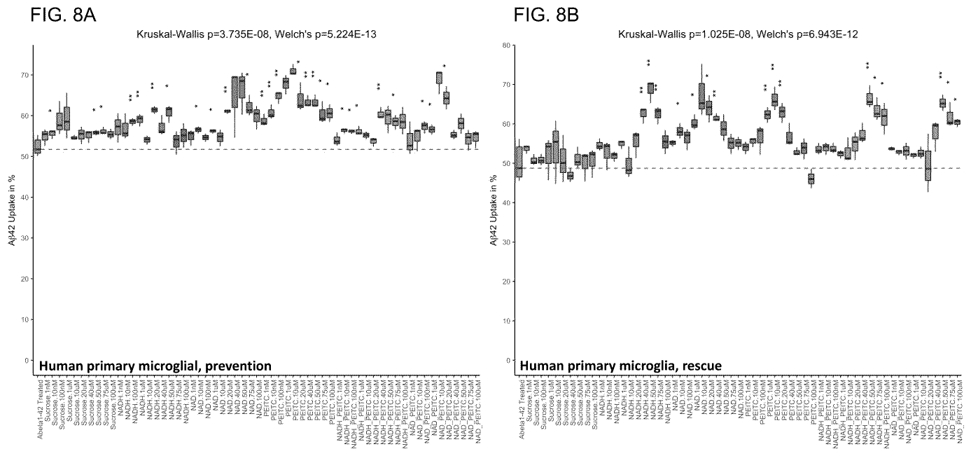

There is considerable interest in microglia-targeted therapies for AD, in part due to the phagocytotic functionality of these cells that can promote neuronal recovery and/or repair. Indeed, phagocytosis of insoluble Aβ by microglia can facilitate CNS clearance of Aβ aggregates and limit formation of amyloid plaques (Ronaldson & Davis, 2020). Indeed, Aβ can bind to receptors at the microglial cell surface, an event that can trigger release of proinflammatory cytokines such as TNF-α and IL-1β, factors that are known to cause neuronal damage (Wang et al. 2015). Additionally, Jiang and colleagues discovered that triggering receptor expressed in myeloid cells 1 (TREM1) can promote Aβ phagocytosis by microglia (Jiang et al. 2016). This study was compelling since genetic knockdown of TREM1 resulted in increased CNS levels of Aβ1-42 in the APP/PSEN1 mouse model of AD. In contrast, selective overexpression of TREM1 on microglia attenuated AD neuropathological and promoted neurocognitive improvement (Jiang et al. 2016). To determine if the natural compounds individually or in combination could promote phagocytosis in microglia, we designed prevention and rescue experiments to measure Aβ1-42 uptake in primary cultures of human microglia via intracellular fluorescence (Online method). Cultured human microglia were exposed to 1 nM, 10 nM, 100 nM, 1 μM, 10 μM, 20 μM, 25 μM, 50 μM, 75 μM, and 100 μM of each compound treatment either individually or in combination with equimolar in both prevention and rescue experiment settings. In the prevention setting, we treated the cultured microglial medium with compounds at Oh. After 24 h, we added 1 μM of Aβ1-42 aggregates to the treated media and measured cell viability at 72 h using the Aβ uptake assay (Online Method, FIG. 8A). The results showed that 27 out of total 50 treatment conditions (5 formulations×10 dosages) significantly increased Aβ1-42 uptake by microglia as compared to the negative control (i.e., microglial cells that were exposed to Aβ1-42 without any compounds). Of particular significance and interest, we observed that PEITC alone stimulated microglial phagocytosis of Aβ1-42 under all tested dosages from 1 nM to 100 μM. NADH significantly increased microglial phagocytosis of Aβ1-42 at 100 nM, 1 μM, 20 μM and 50 μM. NAD+ significantly increased microglial Aβ1-42 uptake at 10 nM, 1 μM, 20 μM and 75 μM. Interestingly, the equimolar combination of NADH and PEITC enabled a lower minimal effective dosage at 10 nM comparing to NADH alone. The equimolar combination of NAD+ and PEITC showed a range of effective dosage ranges from 100 nM, 1 μM, 10 μM and 20 μM. In the rescue experiment settings, primary cultures of human microglia were first challenged with Aβ1-42 for 24 h. At 24 h, compound formulations were added to the culture media containing Aβ1-42. Uptake of Aβ1-42 was then measured 24 h later (i.e., at the 48 h experimental time point, FIG. 8B). The results showed that three and eight dosages of NADH and NAD+ significantly rescued microglial phagocytosis of Aβ1-42 respectively comparing to the negative control condition (NADH: 40, 50, 75 μM, and NAD+: 1 nM to 50 μM). PEITC significantly increased the microglial phagocytosis of Aβ1-42 at 10 nM, 1 μM, 10 μM, 2 0μM and 40 μM. The equimolar formulation of NADH/PEITC and NAD+/PEITC significantly stimulated microglial phagocytosis at 40, 50, 75 and 100 μM, and 50, 75, 100 μM respectively.

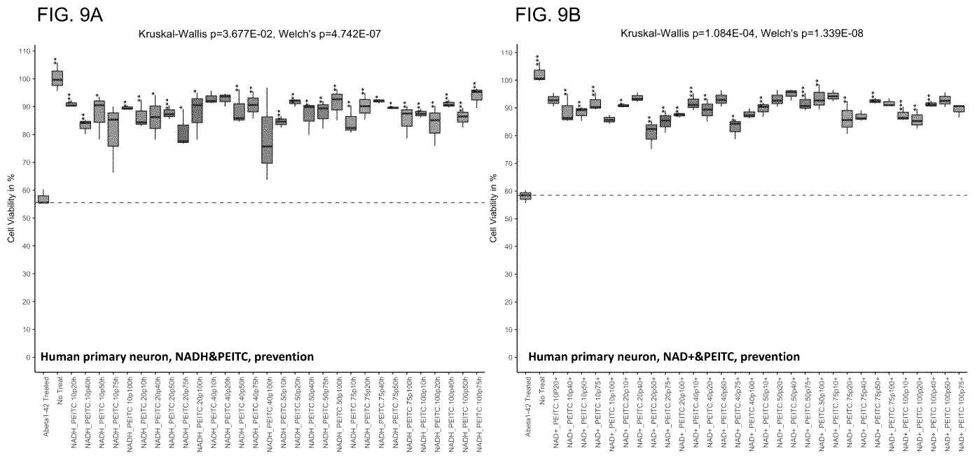

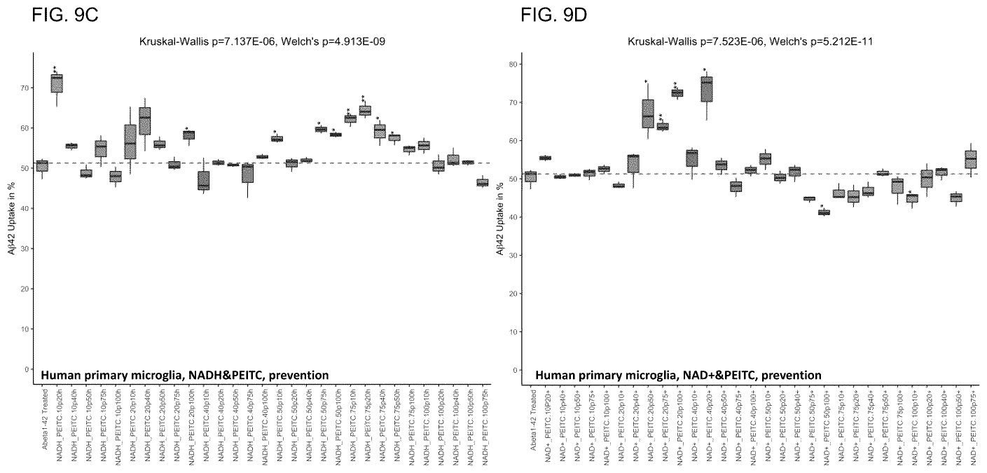

The two equimolar formulations of NADH/PEITC and NAD+/PEITC showed synergistic effects to prevent and rescue neuronal death caused by insoluble Aβ1-42. To determine the optimal dosage of the two formulations for neuronal rescue and microglial phagocytosis stimulation, we treated the primary cultures of human neurons and primary cultures of human microglia in the prevention settings respectively with exhaustive combination of individual dosage of 10 μM, 20 μM, 40 μM, 50 μM, 75 μM, and 100 μM. The results showed that the maximal prevention of neuronal loss can be achieved by the combination of 20 μM PEITC with 20 μM NADH (mean=96.4%, FIG. 9A), and 50 μM PEITC with 40 μM NAD+ (mean=95%, FIG. 9B) respectively, comparing to the negative control (mean=58.1%). The maximal stimulation of microglial phagocytosis of Aβ1-42 can be achieved by the combination of 10 μM PEITC with 20 μM NADH (mean=70.6%, FIG. 9C) and 40 μM PEITC NAD+ with 20 μM NAD+ (mean=72.8%, FIG. 9D) respectively, comparing to the negative control (mean=50.3%).

### Experimental Conclusion

Taken together, we have used unique translational systems biology drug discovery pipeline to identify natural product compounds (i.e., NADH, NAD+, PEITC) that could be directed towards preclinical evaluation as AD therapeutics. In vitro assays for the chosen compounds have demonstrated that they have positive effects against Aβ1-42-mediated cytotoxicity in primary cultures of human neurons and can promote phagocytosis of Aβ1-42 by primary cultures of human microglia. The experimental design included both pre- and post-compound exposure conditions, which effectively modelled preventative and therapeutic situations that are encountered during aging and/or AD pathology. Indeed, the identification of these natural product compounds demonstrated the power of the computational approach. Compared to previous treatment strategies for AD that have failed when subjected to the rigors of clinical trials, we are confident that the repurposing strategy will yield effective therapeutics due to the consideration of several biological pathways and the ability to modify the physiology of multiple CNS cell types.

### Example II

This example describes the synthesis of PEITC analogs (see scheme 1).

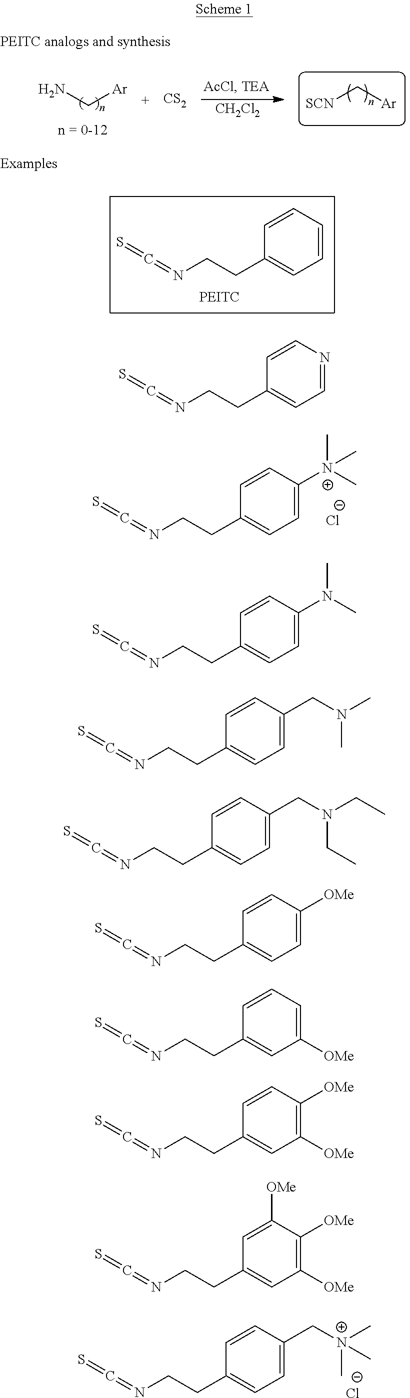

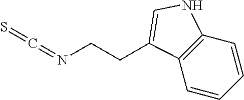 [SMILES: S=C=NCCc1c[nH]c2ccccc12]

The structures contain isothiocynate and aromatic ring connected by (CH₂)n (n=0-12). Aromatic refers to: phenyl and fused phenyl ring and the rings tethering substituent groups such as alkyl, aryl, Cl, Br, F, I, amino, OH, alkoxy, etc and heteroaromatics such as pyridine, indole, quinoline etc and these heteroaromatics attaching substituent groups such as alkyl, aryl, Cl, Br, F, I, amino, OH, alkoxy, etc. Some selected examples are provided shown below. We have purchased and/or made some of these compounds for biological activities. They can be prepared by reaction of corresponding amines with CS₂ in the presence of acetyl chloride (AcCl) and TEA (triethyl amine).

Additional experiments will be conducted with these PEITC analogs using high-throughout screening with MTT and Abeta uptake assays to assess the therapeutic efficacy of such PEITC analogs in the treatment of neurodegenerative disorders as described herein.

Additional in vivo experiments will be conducted with these PEITC analogs to assess the therapeutic efficacy of such PEITC analogs in the treatment of neurodegenerative disorders as described herein.

### Example III

Experiments will be conducted with a purpose of investigating, in vivo, effects of therapeutic combinations of NAD+/PEITC and NADH/PEITC on biomarkers associated Alzheimer's disease (i.e., amyloid beta deposition, accumulation of phosphorylated tau) and neurocognitive performance. These studies will be conducted in two transgenic mouse models of Alzheimer's disease (i.e., 3xTg mice and hTau mice). The 3xTg mouse model contains three mutated human transgenes that are associated with familial Alzheimer's disease (i.e., APP Swedish, MAPT P301L, PSEN1 M146V). These mice display progressive amyloid beta deposition that can be detected in some brain regions by 3-4 months of age. Additionally, tau hyperphosphorylation is present in the hippocampus of these mice by 12-15 months of age. The hTau mouse model has been designed to express only human tau isoforms. As these mice age, they express six isoforms of human tau and develop tau pathology in the CNS. In such experiments, transgenic Alzhemer's disease mice will be treated with increasing dose combinations of NAD+/PEITC and NADH/PEITC via intraperitoneal and oral routes via an once-daily dosing paradigm for 28 consecutive days. Levels of amyloid beta and hyperphosphorylated tau as well as biomarkers associated with neuroinflammation and oxidative stress will be measured using state-of-the-art ELISA technology. Neurocognitive performance in these mice will be evaluated using our conditioned place preference (CPP) methodology. Additionally, pharmacokinetic studies will be performed on NAD+/PEITC and NADH/PEITC dosage combinations following oral and intravenous dosing. Brain and plasma concentrations of NAD+, NADH, and PEITC will be measured using LC/MS-MS.

It is expected that both NAD+/PEITC and NADH/PEITC will decrease brain expression of protein biomarkers relevant to Alzheimer's disease and improve neurocognitive performance as measured by the CPP paradigm. It is also expected that brain permeation of NAD+, NADH, and PEITC will occur at therapeutically effective concentrations as shown by the pharmacokinetic studies.

### INCORPORATION BY REFERENCE

The entire disclosure of each of the patent documents and scientific articles referred to herein is incorporated by reference for all purposes.

The following references denoted throughout the application are incorporated by references in their entireties:
Cunnane, S. C. et al. Brain energy rescue: an emerging therapeutic concept for neurodegenerative disorders of ageing. Nature Reviews Drug Discovery (2020).
Collaborators GBDD. Global, regional, and national burden of Alzheimer's disease and other dementias, 1990-2016: a systematic analysis for the Global Burden of Disease Study 2016. Lancet Neurol. 18, 88-106 (2019).
Naseri, N. N. et al. The complexity of tau in Alzheimer's disease. Neuroscience Letters 705, 183-194 (2019).
Lane, C. A., Hardy, J., & Schott, J. M. Alzheimer's disease. Eur J Neurol. 25, 59-70 (2018).
Jaeger, L. B. et al. Testing the neurovascular hypothesis of Alzheimer's disease: LRP-1 antisense reduces blood-brain barrier clearance, increases brain levels of amyloid-beta protein, and impairs cognition. J Alzheimers Dis. 17, 553-70 (2009).
Ronaldson, P. T., & Davis, T. P. Regulation of blood-brain barrier integrity by microglia in health and disease: A therapeutic opportunity. (forthcoming).
Akhmetzyanova, E., Kletenkov, K., Mukhamedshina, Y., & Rizvanov, A. Different Approaches to Modulation of Microglia Phenotypes After Spinal Cord Injury. Front Syst Neurosci. 13, 37 (2019).
Bard, F. et al. Peripherally administered antibodies against amyloid beta-peptide enter the central nervous system and reduce pathology in a mouse model of Alzheimer disease. Nature Medicine 6, 916-919 (2000).
Salloway, S. et al. Two phase 3 trials of Bapineuzumab in mild-to-moderate Alzheimer's disease. The New England Journal of Medicine 370, 322-333 (2014).
Vandenberghe, R. et al. Bapineuzumab for mild to moderate Alzheimer's disease in two global, randomized, phase 3 trials. Alzheimer's Research & Therapy 8 (2016).
Doody, R. et al. A Phase 3 Trial of Semagacestat for Treatment of Alzheimer's Disease. The New England Journal of Medicine 369, 341-350 (2013).
Timmers, M. et al. Pharmacodynamics of atabecestat (JNJ-54861911), an oral BACE1 inhibitor in patients with early Alzheimer's disease: randomized, double-blind, placebo-controlled study. Alzheimer's Research & Therapy 10, 85 (2018).
Novak, G. et al. Long-term safety and tolerability of atabecestat (JNJ-54861911), an oral BACE1 inhibitor, in early Alzheimer's disease spectrum patients: a randomized, double-blind, placebo-controlled study and a two-period extension study. Alzheimer's Research & Therapy 12, (2020).
Kennedy, M. E. et al. The BACE1 inhibitor verubecestat (MK-8931) reduces CNS β-amyloid in animal models and in Alzheimer's disease patients. Science Translational Medicine 8, 363ra150 (2016).
Egan, M. F. et al. Randomized trial of verubecestat for prodromal Alzheimer's disease. The New England Journal of Medicine 380, 1408-1420 (2019a).
Egan, M. F. et al. Further analyses of the safety of verubecestat in the phase 3 EPOCH trial of mild-to-moderate Alzheimer's disease. Alzheimer's Research & Therapy 11, 68 (2019b).
Petyuk, V. A. et al. The human brainome: network analysis identifies HSPA2 as a novel Alzheimer's disease target. Brain 141, 2721-2739 (2018).
Patel, K. R. et al. Single Cell-type Integrative Network Modeling Identified Novel Microglial-specific Targets for the Phagosome in Alzheimer's Disease. Preprint at https://www.biorxiv.org/content/10.1101/2020.06.09.143529v1 (2020).

The following additional references are incorporated by reference in their entireties:
Kim, T., C. S. Lim, and B. K. Kaang, Cell type-specific gene expression profiling in brain tissue: comparison between TRAP, LCM and RNA-seq. BMB Rep, 2015. 48(7): p. 388-94.
Zhao, Y., et al., TREM2 Is a Receptor for beta-Amyloid that Mediates Microglial Function. Neuron, 2018. 97(5): p. 1023-1031 e7.
Rajendran, L. and R. C. Paolicelli, Microglia-Mediated Synapse Loss in Alzheimer's Disease. J Neurosci, 2018. 38(12): p. 2911-2919.
Condello, C., P. Yuan, and J. Grutzendler, Microglia-Mediated Neuroprotection, TREM2, and Alzheimer's Disease: Evidence From Optical Imaging. Biol Psychiatry, 2018. 83(4): p. 377-387.
Sims, R., et al., Rare coding variants in PLCG2, ABI3, and TREM2 implicate microglial-mediated innate immunity in Alzheimer's disease. Nat Genet, 2017. 49(9): p. 1373-1384.
Zeisel, A., et al., Brain structure. Cell types in the mouse cortex and hippocampus revealed by single-cell RNA-seq. Science, 2015. 347(6226): p. 1138-42.
Zhang, Y., et al., An RNA-sequencing transcriptome and splicing database of glia, neurons, and vascular cells of the cerebral cortex. J Neurosci, 2014. 34(36): p. 11929-47.
Rui Chang, M. A., Kwangsik Won, Gabi Kastermuelle, Andrew Sarkin, Rima Kaddurah-Daouk. Predictive Network Modeling identifies Sex-specific Metabolic Drivers in Alzheimer's Disease. in Alzheimer's & Dementia (In Submission). 2018.
Canevelli, M., et al., Sex and gender differences in the treatment of Alzheimer's disease: A systematic review of randomized controlled trials. Pharmacol Res, 2017. 115: p. 218-223.
Zhao, L., et al., Sex differences in metabolic aging of the brain: insights into female susceptibility to Alzheimer's disease. Neurobiol Aging, 2016. 42: p. 69-79.
Ronquillo, J. G., M. R. Baer, and W. T. Lester, Sex-specific patterns and differences in dementia and Alzheimer's disease using informatics approaches. J Women Aging, 2016. 28(5): p. 403-11.
Mazure, C. M. and J. Swendsen, Sex differences in Alzheimer's disease and other dementias. Lancet Neurol, 2016. 15(5): p. 451-2.
Laws, K. R., K. Irvine, and T. M. Gale, Sex differences in cognitive impairment in Alzheimer's disease. World J Psychiatry, 2016. 6(1): p. 54-65.
Munro, C. A., Sex differences in Alzheimer's disease risk: are we looking at the wrong hormones? Int Psychogeriatr, 2014. 26(10): p. 1579-84.
Zhang, B., et al., Integrated systems approach identifies genetic nodes and networks in late-onset Alzheimer's disease. Cell, 2013. 153(3): p. 707-20.
Chang, R., R. Shoemaker, and W. Wang, Systematic search for recipes to generate induced pluripotent stem cells. PLoS Comput Biol, 2011. 7(12): p. e1002300.
Chang, R., R. Shoemaker, and W. Wang, A novel knowledge-driven systems biology approach for phenotype prediction upon genetic intervention. IEEE/ACM Trans Comput Biol Bioinform, 2011. 8(5): p. 1170-82.
Chang, R., J. R. Karr, and E. E. Schadt, Causal inference in biology networks with integrated belief propagation. Pac Symp Biocomput, 2015: p. 359-70.
Carcamo-Orive, I., et al., Analysis of Transcriptional Variability in a Large Human iPSC Library Reveals Genetic and Non-genetic Determinants of Heterogeneity. Cell Stem Cell, 2017. 20(4): p. 518-532.
Zissimopoulos, J. M., et al., Sex and Race Differences in the Association Between Statin Use and the Incidence of Alzheimer Disease. JAMA Neurol, 2017. 74(2): p. 225-232.
Chen, Y. Q., et al., Variations in DNA elucidate molecular networks that cause disease. Nature, 2008. 452(7186): p. 429-435.
Schadt, E. E., Molecular networks as sensors and drivers of common human diseases. Nature, 2009. 461(7261): p. 218-223.
Schadt, E. E. and J. L. Bjorkegren, NEW: network-enabled wisdom in biology, medicine, and health care. Sci Transl Med, 2012. 4(115): p. 115rv1.
Gandy, S., et al., CR1 and the “vanishing amyloid” hypothesis of Alzheimer's disease. Biol Psychiatry, 2013. 73(5): p. 393-5.
Rui Chang*#, K. Z., Kruti Patel, Marc R Henrion, Sara Moein, Min Tang, Philip De Jager, Elizabeth Bradshaw, Predictive Network Analysis Identified Novel Therapeutic Targets in Microglials for Alzheimer's diseases. Nature Genetics (In Submission), 2018.
Ivan Carcamo-Orive, M. Y. R. H., Kuixi Zhu, Paige Cundiff, Noam D. Beckmann, Sunita D'Souza, Eric E. Schadt, Thomas Quertermous, Joshua W. Knowles, Rui Chang, Predictive network modeling in human induced pluripotent stem cells identifies key driver genes for insulin responsiveness. Nature Genetics (In Submission), 2018.
Rui Chang, W. W. Novel algorithm for Bayesian network parameter learning with informative prior constraints. in Neural Networks (IJCNN), The 2010 International Joint Conference on. 2010.
Toledo, J. B., et al., Metabolic Network Failures in Alzheimer Disease—A Biochemical Roadmap. Alzheimer's & Dementia, 2017.
Cohain, A., et al., Exploring the Reproducibility of Probabilistic Causal Molecular Network Models. Pac Symp Biocomput, 2016. 22: p. 120-131.
Myers, A., et al., THE HUMAN BRAINOME: HUMAN BRAIN GENOME, TRANSCRIPTOME, AND PROTEOME INTEGRATION, in Alzheimer's & Dementia: The Journal of the Alzheimer's Association. 2016. p. 237-238.
Zhao, X., et al., Resolving complex structural genomic rearrangements using a randomized approach. Genome Biol, 2016. 17(1): p. 126.
Chen, X., et al., Manta: rapid detection of structural variants and indels for germline and cancer sequencing applications. Bioinformatics, 2016. 32(8): p. 1220-2.
Layer, R. M., et al., LUMPY: a probabilistic framework for structural variant discovery. Genome Biol, 2014. 15(6): p. R84.
Pinto, D., et al., Comprehensive assessment of array-based platforms and calling algorithms for detection of copy number variants. Nat Biotechnol, 2011. 29(6): p. 512-20.
Colella, S., et al., QuantiSNP: an Objective Bayes Hidden-Markov Model to detect and accurately map copy number variation using SNP genotyping data. Nucleic Acids Res, 2007. 35(6): p. 2013-25.
Wang, K., et al., PennCNV: an integrated hidden Markov model designed for high-resolution copy number variation detection in whole-genome SNP genotyping data. Genome Res, 2007. 17(11): p. 1665-74.
Darmanis, S., et al., A survey of human brain transcriptome diversity at the single cell level. Proc Natl Acad Sci USA, 2015. 112(23): p. 7285-90.
Carrillo-de Sauvage, M. A., et al., Potent and multiple regulatory actions of microglial glucocorticoid receptors during CNS inflammation. Cell Death Differ, 2013. 20(11): p. 1546-57.
Nadra, K., et al., Phosphatidic acid mediates demyelination in Lpin1 mutant mice. Genes Dev, 2008. 22(12): p. 1647-61.
Bana, L., et al., Liposomes bi-functionalized with phosphatidic acid and an ApoE-derived peptide affect Abeta aggregation features and cross the blood-brain-barrier: implications for therapy of Alzheimer disease. Nanomedicine, 2014. 10(7): p. 1583-90.
Sivendran, S., et al., Dissection of immune gene networks in primary melanoma tumors critical for antitumor surveillance of patients with stage II-III resectable disease. J Invest Dermatol, 2014. 134(8): p. 2202-11.
Xie, Z. R. and M. J. Hwang, Ligand-binding site prediction using ligand-interacting and binding site-enriched protein triangles. Bioinformatics, 2012. 28(12): p. 1579-85.
Xie, Z. R. and M. J. Hwang, Methods for predicting protein-ligand binding sites. Methods Mol Biol, 2015. 1215: p. 383-98.
Xie, Z. R., et al., LISE: a server using ligand-interacting and site-enriched protein triangles for prediction of ligand-binding sites. Nucleic Acids Res, 2013. 41(Web Server issue): p. W292-6.
Luco, J. M., Prediction of the brain-blood distribution of a large set of drugs from structurally derived descriptors using partial least-squares (PLS) modeling. J Chem Inf Comput Sci, 1999. 39(2): p. 396-404.
Subramanian, A., et al., A Next Generation Connectivity Map: L1000 Platform and the First 1,000,000 Profiles. Cell, 2017. 171(6): p. 1437-1452 e17.
Eto, K., et al., Role of NADH shuttle system in glucose-induced activation of mitochondrial metabolism and insulin secretion. Science, 1999. 283(5404): p. 981-5.
Stein, L. R. and S. Imai, The dynamic regulation of NAD metabolism in mitochondria. Trends Endocrinol Metab, 2012. 23(9): p. 420-8.
Pearl, S. M., et al., Effects of NADH on dopamine release in rat striatum. Synapse, 2000. 36(2): p. 95-101.
Vrecko, K., et al., NADH stimulates endogenous dopamine biosynthesis by enhancing the recycling of tetrahydrobiopterin in rat phaeochromocytoma cells. Biochim Biophys Acta, 1997. 1361(1): p. 59-65.
Birkmayer, J. G., et al., Nicotinamide adenine dinucleotide (NADH)—a new therapeutic approach to Parkinson's disease. Comparison of oral and parenteral application. Acta Neurol Scand Suppl, 1993. 146: p. 32-5.
Forsyth, L. M., et al., Therapeutic effects of oral NADH on the symptoms of patients with chronic fatigue syndrome. Ann Allergy Asthma Immunol, 1999. 82(2): p. 185-91.
Birkmayer, J. G., Coenzyme nicotinamide adenine dinucleotide: new therapeutic approach for improving dementia of the Alzheimer type. Ann Clin Lab Sci, 1996. 26(1): p. 1-9.
Demarin, V., et al., Treatment of Alzheimer's disease with stabilized oral nicotinamide adenine dinucleotide: a randomized, double-blind study. Drugs Exp Clin Res, 2004. 30(1): p. 27-33.
Ghareghani, M., et al., Fluvoxamine stimulates oligodendrogenesis of cultured neural stem cells and attenuates inflammation and demyelination in an animal model of multiple sclerosis. Sci Rep, 2017. 7(1): p. 4923.
Ryan, K. J., et al., A human microglia-like cellular model for assessing the effects of neurodegenerative disease gene variants. Sci Transl Med, 2017. 9(421).
Elyaman, W., et al., BAD and Bcl-2 regulation are early events linking neuronal endoplasmic reticulum stress to mitochondria-mediated apoptosis. Brain Res Mol Brain Res, 2002. 109(1-2): p. 233-8.
Elyaman, W., C. Yardin, and J. Hugon, Involvement of glycogen synthase kinase-3beta and tau phosphorylation in neuronal Golgi disassembly. J Neurochem, 2002. 81(4): p. 870-80.
Maia, L. F., et al., Changes in amyloid-beta and Tau in the cerebrospinal fluid of transgenic mice overexpressing amyloid precursor protein. Sci Transl Med, 2013. 5(194): p. 194re2.
Radde, R., et al., Abeta42-driven cerebral amyloidosis in transgenic mice reveals early and robust pathology. EMBO Rep, 2006. 7(9): p. 940-6.
Chang, R., Zhu Kuixi, Arnold Matthias, Kwangsik Nho, Henrion Marc, Moein Sara, Blach Collete, Tenenbaum J D, Kastenmueller G, Saykin A J, Eugenia T, Brinton Roberta, Kaddurah-Daouk Raduk, Predictive Network Analysis Identified Sex, Genotype and Clinic-stage specific Metabolite Fuels for Alzheimer's disease. Nature Metabolism (In Submission), 2018.
Bush, E. C., et al., PLATE-Seq for genome-wide regulatory network analysis of high-throughput screens. Nat Commun, 2017. 8(1): p. 105.
Harold, D., et al., Genome-wide association study identifies variants at CLU and PICALM associated with Alzheimer's disease. Nat Genet, 2009. 41(10): p. 1088-93.
Lambert, J. C., et al., Genome-wide association study identifies variants at CLU and CR1 associated with Alzheimer's disease. Nat Genet, 2009. 41(10): p. 1094-9.
Bu, G., Apolipoprotein E and its receptors in Alzheimer's disease: pathways, pathogenesis and therapy. Nat Rev Neurosci, 2009. 10(5): p. 333-44.
Kanekiyo, T., H. Xu, and G. Bu, ApoE and Abeta in Alzheimer's disease: accidental encounters or partners? Neuron, 2014. 81(4): p. 740-54.
Castellano, J. M., et al., Human apoE isoforms differentially regulate brain amyloid-beta peptide clearance. Sci Transl Med, 2011. 3(89): p. 89ra57.
Sakae, N., et al., ABCA7 Deficiency Accelerates Amyloid-beta Generation and Alzheimer's Neuronal Pathology. J Neurosci, 2016. 36(13): p. 3848-59.
Shinohara, M., et al., APOE2 eases cognitive decline during aging: clinical and preclinical evaluations. Ann Neurol, 2016.
Liu, C. C., et al., Deficiency in LRP6-mediated Wnt signaling contributes to synaptic abnormalities and amyloid pathology in Alzheimer's disease. Neuron, 2014. 84(1): p. 63-77.
Dieterle, F., et al., Renal biomarker qualification submission: a dialog between the FDA-EMEA and Predictive Safety Testing Consortium. Nat Biotechnol, 2010. 28(5): p. 455-62.
Tachibana, M., et al., Rescuing effects of RXR agonist bexarotene on aging-related synapse loss depend on neuronal LRP1. Exp Neurol, 2016. 277: p. 1-9.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating a mammal suffering from a condition characterized with neuronal cell death, dysfunctional energetic function, unregulated microglia phagocytic activity, and/or other related de-regulated biological functions comprising
administering to the mammal a composition comprising one or more agents capable of protecting neurons from cell death and unregulated microglia phagocytic activity,
wherein the agent is selected from PEITC, an analog of PEITC, NAD+, NADH, NMN, NR, 7-methylsulfinylheptyl, 8-methylsulfinyloctyl, vitamin c, glutathione, and myrosinase,
wherein the condition is one or more of a neurodegenerative disorder, aging, systemic inflammation, neuroinflammation, cancer, and diabetes,
wherein the mammal is a human patient.

2. (canceled)

3. The method of claim 1, wherein the neurodegenerative disorder selected from AD, Parkinson's disease, Huntington's disease, amyotrophic lateral sclerosis, motor neuron disease, subjective memory complaints, and mild cognitive impairment (MCI). In some embodiments, the AD is an early stage, prodromal phase of AD or late stage.

4. (canceled)

5. (canceled)

6. (canceled)

7. The method of claim 1, wherein the PEITC analog is selected from the group consisting of: 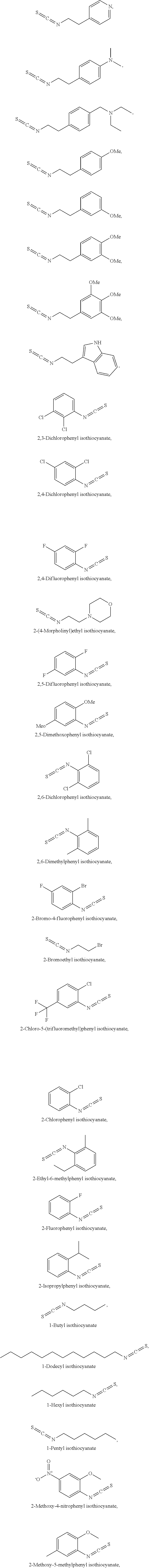 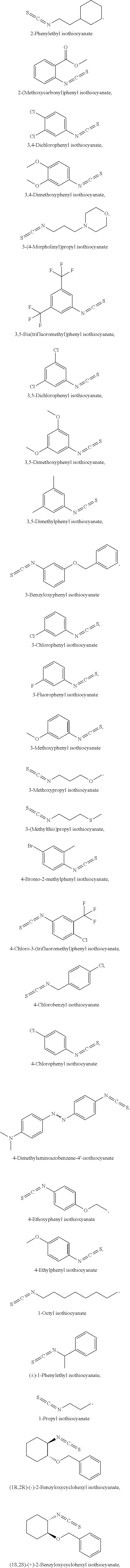 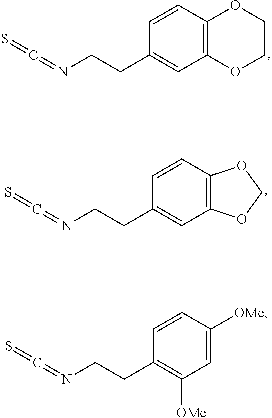 [SMILES: S=C=NCCc1ccc2c(c1)OCCO2 ; S=C=NCCc1ccc2c(c1)OCO2 ; COc1ccc(CCN=C=S)c(OC)c1]
Allylglucosinolate (sinigrin), allyl isothiocyanate, Benzylglucosinolate (Glucotropaeolin), benzyl isothiocyanate, Gluconasturtiin, (R)-4-(methylsulfinyl)butylglucosinolate (Glucoraphanin), (R)-4-(methylsulfinyl)butyl isothiocyanate (sulforaphane), (R)-2-hydroxybut-3 -enylglucosinolate (progoitrin), (S)-5-vinyloxazolidine-2-thione (goitrin), and any chemical moiety related to watercress and/or other cruciferous plant extraction.

8. The method of claim 1,
wherein the agent capable of protecting neurons from cell death and unregulated microglia phagocytic activity is derived from natural plants and seeds, and their extracts or derivatives;
wherein the agent capable of protecting neurons from cell death and unregulated microglia phagocytic activity is derived from watercress, Cruciferous Vegetables, mustard, white mustard (Sinapis alba), garden cress (Lepidium sativum), wasabi (Wasabia japonica), and daikon (Raphanus sativus); or
wherein the agent capable of protecting neurons from cell death and unregulated microglia phagocytic activity is derived from members of the family Brassicaceae, including yellow mustard (Brassica juncea), rape seed (Brassica napus), and common dietary Brassicas including, but not limited to, broccoli, cauliflower, cabbage, bok choy, kale, Papaya seeds, and cabbage aphid.

9-30. (canceled)

31. A method of treating, preventing and/or ameliorating symptoms of a neurodegenerative disorder in a mammal in need thereof, the method comprising
administering to the mammal an effective amount of an agent capable of protecting neurons from cell death and unregulated microglia phagocytic activity,
wherein the agent is a pharmaceutical composition comprising a therapeutically effective amount of one or more of PEITC, an analog of PEITC, NAD+, NADH, NMN, NR, 7-methylsulfinylheptyl, 8-methylsulfinyloctyl, vitamin c, glutathione, and myrosinase,
wherein the neurodegenerative disorder is selected from AD, Parkinson's disease, Huntington's disease, amyotrophic lateral sclerosis, motor neuron disease, subjective memory complaints, and MCI,
wherein the mammal is a human patient.

32-35. (canceled)

36. The method of claim 31, wherein the PEITC analog is selected from the group consisting of: 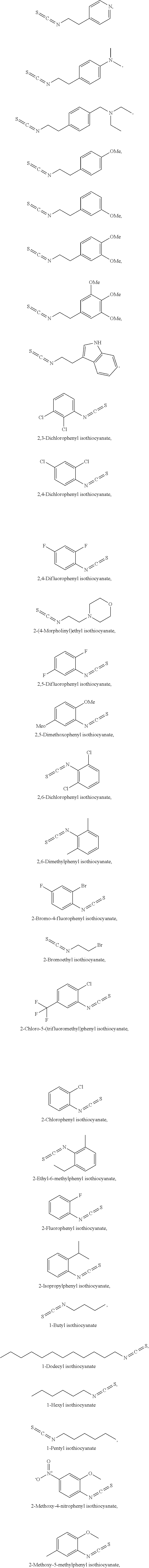 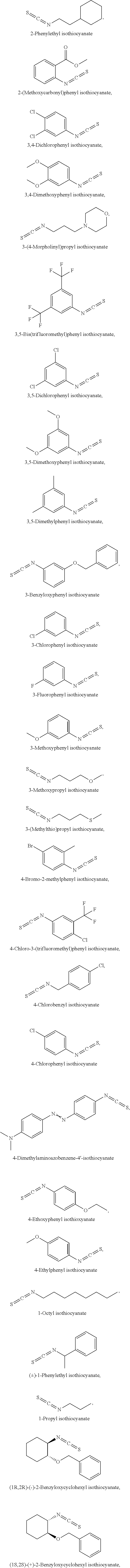 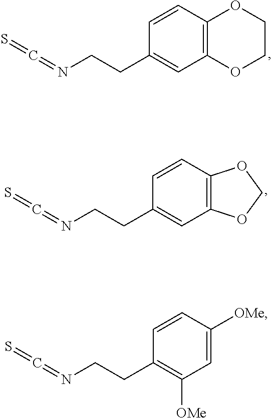 [SMILES: S=C=NCCc1ccc2c(c1)OCCO2 ; S=C=NCCc1ccc2c(c1)OCO2 ; COc1ccc(CCN=C=S)c(OC)c1]
Allylglucosinolate (sinigrin), allyl isothiocyanate, Benzylglucosinolate (Glucotropaeolin), benzyl isothiocyanate, Gluconasturtiin, (R)-4-(methylsulfinyl)butylglucosinolate (Glucoraphanin), (R)-4-(methylsulfinyl)butyl isothiocyanate (sulforaphane), (R)-2-hydroxybut-3 -enylglucosinolate (progoitrin), (S)-5-vinyloxazolidine-2-thione (goitrin), and any chemical moiety related to watercress and/or other cruciferous plant extraction.

37. The method of claim 31,
wherein the agent is derived from natural plants and seeds, and their extracts or derivatives, and/or
wherein the agent is derived from watercress, Cruciferous Vegetables, mustard, white mustard (Sinapis alba), garden cress (Lepidium sativum), wasabi (Wasabia japonica), and daikon (Raphanus sativus), and/or
wherein the agent is derived from members of the family Brassicaceae, including yellow mustard (Brassica juncea), rape seed (Brassica napus), and common dietary Brassicas including, but not limited to, broccoli, cauliflower, cabbage, bok choy, kale, Papaya seeds, and cabbage aphid.

38-41. (canceled)

42. A composition comprising one or more of the following: PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase.

43. The composition of claim 42, wherein the PEITC analog is selected from the group consisting of: 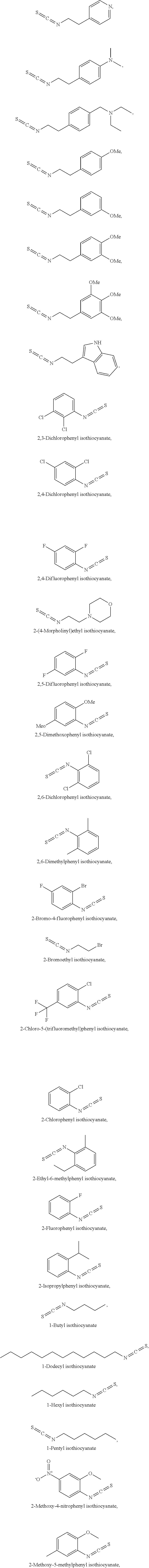 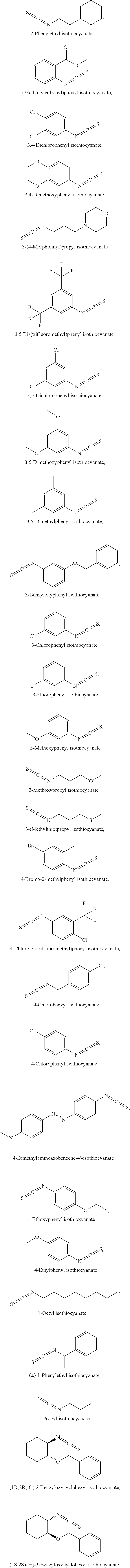 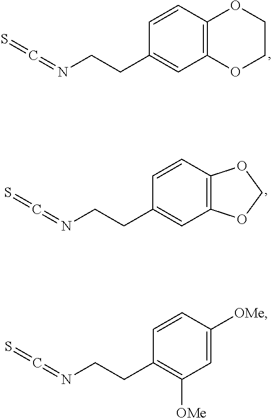 [SMILES: S=C=NCCc1ccc2c(c1)OCCO2 ; S=C=NCCc1ccc2c(c1)OCO2 ; COc1ccc(CCN=C=S)c(OC)c1]
Allylglucosinolate (sinigrin), allyl isothiocyanate, Benzylglucosinolate (Glucotropaeolin), benzyl isothiocyanate, Gluconasturtiin, (R)-4-(methylsulfinyl)butylglucosinolate (Glucoraphanin), (R)-4-(methylsulfinyl)butyl isothiocyanate (sulforaphane), (R)-2-hydroxybut-3 -enylglucosinolate (progoitrin), (S)-5-vinyloxazolidine-2-thione (goitrin), and any chemical moiety related to watercress and/or other cruciferous plant extraction.

44. The composition of claim 42, wherein the agent capable of protecting neurons from cell death and unregulated microglia phagocytic activity is derived from natural plants and seeds, and their extracts or derivatives.

45. The composition of claim 42, wherein the agent capable of protecting neurons from cell death and unregulated microglia phagocytic activity is derived from watercress, Cruciferous Vegetables, mustard, white mustard (Sinapis alba), garden cress (Lepidium sativum), wasabi (Wasabia japonica), and daikon (Raphanus sativus).

46. The composition of claim 42, wherein the agent capable of protecting neurons from cell death and unregulated microglia phagocytic activity is derived from members of the family Brassicaceae, including yellow mustard (Brassica juncea), rape seed (Brassica napus), and common dietary Brassicas including, but not limited to, broccoli, cauliflower, cabbage, bok choy, kale, Papaya seeds, and cabbage aphid.

47. The composition of claim 42, wherein the composition comprises two or more of: PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase.

48. The composition of claim 42, wherein the composition comprises three or more of: PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase.

49. The composition of claim 42, wherein the composition comprises: PEITC, an analog of PEITC, NAD+, NADH, NMN, NR, a chemical moiety comprising isothiocyanate (e.g., 7-methylsulfinylheptyl, 8-methylsulfinyloctyl), vitamin c (ascorbate acid), glutathione, and myrosinase.

50. The composition of claim 42, wherein the composition is an over-the-counter composition, or a pharmacological prescription.

51-64. (canceled)
